(19) United States
(12) Patent Application Publication
(10) Pub. No.: US 2025/0157614 A1
(43) Pub. Date: May 15, 2025

(54) SUBSTANCE DELIVERY PLANNING SYSTEM

(71) Applicant: Syqe Medical Ltd., Tel-Aviv (IL)

(72) Inventors: Perry DAVIDSON, Safed (IL); Ianiv Yosef EISENSCHER, Tel Aviv-Yafo (IL); Yotam HOFFMANN, Safed (IL)

(73) Assignee: Syqe Medical Ltd., Tel-Aviv (IL)

(21) Appl. No.: 19/026,833

(22) Filed: Jan. 17, 2025

Related U.S. Application Data
(63) Continuation of application No. 17/613,066, filed on Nov. 21, 2021.
(60) Provisional application No. 62/850,580, filed on May 21, 2019.

(51) Int. Cl. G16H 20/13; A61K 36/185

(52) U.S. Cl. CPC G16H 20/13; A61K 36/185

(57) ABSTRACT

Some embodiments relate to a method for generating a delivery regimen of at least one active substance to a user, comprising: receiving input including one or both of timed activities to be engaged by the user, timed preferred user experiences; and automatically generating a delivery regimen including at least one of delivery times and amounts of the at least one active substance, based on: (a) estimating at least one of a timing and magnitude of at least one effect expected to be induced by the at least one active substance on the user; and (b) estimating an impact of the at least one effect on the timed activities and/or the timed preferred user experiences.

### RELATED APPLICATIONS

This application is a continuation of U.S. patent application Ser. No. 17/613,066 filed on Nov. 21, 2021, which is a National Phase of PCT Patent Application No. PCT/IL2020/050559 having International Filing Date of May 21, 2020, which claims the benefit of priority under 35 USC § 119(e) of U.S. Provisional Patent Application No. 62/850,580 filed on May 21, 2019. The contents of the above applications are all incorporated by reference as if fully set forth herein in their entirety.

### FIELD AND BACKGROUND

The present disclosure, in some embodiments thereof, relates to delivery of at least one active substance to a user, and, more particularly, but not exclusively, to a system and method for automatically generating a delivery regimen of the at least one active substance to the user.

PCT Publication WO2016/001922 to Davidson et al. titled “METHODS, DEVICES AND SYSTEMS FOR PULMONARY DELIVERY OF ACTIVE AGENTS” discloses “Provided herein is a method of pulmonary delivering to a subject at least a first pharmacologically active agent and a second pharmacologically active agent, at least one of which being in at least one plant material; the method is carried out by independently delivering the agents to the subject using a metered dose inhaler device that is configured to vaporize at least a first pre-determined vaporized amount of the first agent and at least a second pre-determined vaporized amount of the second agent upon controllably heating the plant material, wherein heating is effected such that the first pre-determined vaporized amount is delivered to the subject successively, concomitantly and/or at least partially overlapping with the delivery of the second pre-determined vaporized amount, and wherein each of the pre-determined vaporized amounts of each of the agents independently induces in the subject at least one pharmacokinetic effect and/or at least one pharmacodynamic effect.” (Abstract).

### SUMMARY

According to an aspect of some embodiments there is provided a method for generating a delivery regimen of at least one active substance to a user, comprising:
receiving input including one or both of timed activities of the user, timed preferred user experiences;
and
automatically generating using a processor a delivery regimen including at least one of delivery times and amounts of the at least one active substance, based on:
(a) estimating at least one of a timing and magnitude of at least one effect expected to be induced by the at least one active substance on the user; and
(b) estimating an impact of the at least one effect on the timed activities and/or the timed preferred user experiences.

In some embodiments, the method comprises controlling a delivery device according to the delivery regimen, to deliver the amounts at the delivery times to a user.

In some embodiments, the method comprises locking the delivery device to prevent or limit delivery of the at least one active substance at one or more times defined according to the generated delivery regimen.

In some embodiments, the method comprises automatically issuing a notification regarding a previous and/or current and/or future status of the at least one effect at one or more time points throughout the delivery regimen.

In some embodiments, the method comprises automatically recommending an activity or action based on the previous and/or current and/or future status of the at least one effect.

In some embodiments, the method comprises storing the generated delivery regimen in correlation with at least one effect induced by the active substance and exhibited during or consequently to executing of the delivery regimen.

In some embodiments, the regimen comprises different time windows within which the effect on the user is within defined limits.

In some embodiments, the limits are defined according to: a type of activity engaged by user, user preferences, and/or use restrictions.

In some embodiments, the method comprises accessing one or both of personal user data and population data, pertaining to pharmacokinetics and/or pharmacodynamics of the at least one active substance, and using the data in generating and/or adjusting the delivery regimen.

In some embodiments, estimating takes into account an expected lasting duration of the at least one effect.

In some embodiments, the at least one active substance influences a psychoactive state of the user.

In some embodiments, the at least one active substance comprises a cannabinoid.

In some embodiments, the preferred user experience includes a desired potency of an effect and/or a type of effect induced by the at least one active substance.

In some embodiments, the type of effect is from the group of analgesic effect, uplifting effect, sedative effect, calming effect, aphrodisiac effect, focus effect, social effect, appetizing effect, creative effect, energizing effect, and euphoric effect.

In some embodiments, the method comprises estimating an expected blood plasma level of the at least one active substance at one or more selected times during or following executing of the delivery regimen.

In some embodiments, the method comprises dynamically modifying the delivery regimen according to indications related to changes in a user's activity schedule, and/or according to sensed data indicative of a user status.

In some embodiments, the method comprises estimating a likelihood of the user to experience a certain psychoactive state at one or more selected times during or following executing of the delivery regimen.

In some embodiments, the method comprises pairing the delivery times with the amounts, wherein the amounts vary at least once.

In some embodiments, the regimen sets delivery of a placebo at one or more of the delivery times.

In some embodiments, the method comprises defining a maximal level of the at least one effect, wherein the delivery regimen is planned such that the at least one effect does not exceed the maximal level.

In some embodiments, delivery comprises pulmonary delivery via a metered dose inhaler device.

According to an aspect of some embodiments there is provided a system for planned delivery of at least one active substance to a user, comprising:
a user interface configured to receive as input at least one of timed activities to be engaged by the user, timed preferred user experiences;
a processor programmed to generate a delivery regimen including at least one of delivery times and amounts of the at least one active substance, based at least partially on:
(a) estimating at least one of a timing and magnitude of at least one effect expected to be induced by the at least one active substance on the user; and
(b) estimating an impact of the at least one effect on the timed activities and/or the timed preferred user experiences;
and
a delivery device operative to deliver to the user the at least one active substance according to the delivery regimen.

In some embodiments, the delivery device is configured to lock to thereby prevent or limit delivery of at the least one active substance at one or more times defined according to the generated delivery regimen.

In some embodiments, the processor is programmed to generate the delivery regimen based on one or both of personal user data and population data, pertaining to pharmacokinetics and/or pharmacodynamics of the at least one active substance.

In some embodiments, the processor is programmed to generate the delivery regimen based on use restrictions.

In some embodiments, the processor and/or the delivery device are in communication with a memory which stores delivery times during which the at least one active substance is delivered to the user via the delivery device.

In some embodiments, the memory stores one or more delivery regimens and/or effects induced by the at least one active substance on the user at one or more times during executing of each of the one or more delivery regimens.

In some embodiments, the processor is in association with a communication module which is in communication with the user's personal digital device.

In some embodiments, the processor is programmed to generate the delivery regimen according to one or more pharmacokinetic limits and/or one or more pharmacodynamic limits.

In some embodiments, the processor is programmed to calculate a cumulative effect of a plurality of delivery events on the user, and to plan at least delivery times and amounts of the at least one active substance according to the cumulative effect.

In some embodiments, the delivery device is an inhaler device.

In some embodiments, the delivery device contains or is structured to be in operable contact with a plurality of dose units comprising known amounts of the at least one active substance, and to actuate use of the dose units according to the delivery regimen in response to an indication from a controller programmed to control and execute the delivery regimen.

In some embodiments, at least one of the dose units comprise botanical material.

In some embodiments, the botanical material comprises cannabis.

In some embodiments, the user interface comprises or is in communication with one or more sensors or devices suitable for assessing an effect of selected delivery events or of a full delivery regimen on the user.

In some embodiments, the user interface notifies a user regarding the user's current pharmacodynamic state and/or current pharmacokinetic state based on the time that passed from one or more previous delivery events.

In some embodiments, the delivery device comprises a controller programmed to lock the delivery device.

In some embodiments, the processor generates the delivery regimen based on an expected interaction of one or both of the timed activities and the timed preferred user experiences with the at least one effect.

In some embodiments, the processor is programmed to generate the delivery regimen based on a tradeoff between a timed activity and a preferred user experience, and to define at least one of the delivery times and amounts according to the tradeoff.

In some embodiments, the delivery regimen includes a plurality of active substances and the processor is programmed to generate the delivery regimen based on the at least one effect selected between: an additive effect, a synergistic effect and a counteractive effect between at least two of the active substances.

According to an aspect of some embodiments there is provided a method for generating a delivery regimen of at least one active substance to a user, comprising:
receiving input including at least part of a user's schedule and at least one preferred user experience during at least a portion of the part of the schedule;
automatically generating a delivery regimen including at least one of delivery times and amounts of the at least one active substance, wherein generating a delivery regimen is at least partially based on at least one effect induced by the at least one active substance on the user.

According to an aspect of some embodiments there is provided a method for generating a delivery regimen of at least one active substance to a user via inhalation, comprising:
receiving input including one or both of timed activities to be engaged by the user, timed preferred user experiences; and
automatically generating a delivery regimen including at least one of delivery times and amounts of the at least one active substance, based on an expected timeline of a blood plasma level of the at least one active substance and based on an estimated impact of the blood plasma level on one or both of the timed activities and the timed preferred user experiences.

According to an aspect of some embodiments there is provided a method for generating time-based recommendations to a user, comprising:
receiving input including a timing and amount of one or more doses of at least one active substance previously taken by the user and/or one or more planned doses; and
automatically generating a time-based recommendation based at least partially on estimating at least one of a timing and magnitude of at least one effect expected to be induced by the at least one active substance on the user, the time-based recommendation including an indication of the user's capability for engaging in one or more activities, wherein the capability is correlated with the at least one effect; and
outputting the recommendation.

In some embodiments, the input includes input obtained by automatically accessing information on a delivery device used for delivering the at least one or more doses previously taken by the user.

In some embodiments, the input includes input received via a user interface.

In some embodiments, the recommendation is set for a pre-defined likelihood of the user being capable to engage an activity.

In some embodiments, the recommendation includes a regimen of future deliveries of the same active substance previously taken by the user.

In some embodiments, the recommendation includes a regimen of future deliveries of a different active substance which is known to affect at least one of the timing and magnitude of the effect of the active substance previously taken by the user.

In some embodiments, delivery is via inhalation.

In some embodiments, the method further comprises controlling a delivery device to deliver the active substance according to the regimen.

According to an aspect of some embodiments a method is provided for generating a delivery regimen of at least one active substance to a user, comprising:
receiving input including one or both of timed activities to be engaged by the user, timed preferred user experiences;
and
automatically generating a delivery regimen including at least one of delivery times and amounts of the at least one active substance, based on:
(a) estimating at least one of a timing and magnitude of at least one effect expected to be induced by the at least one active substance on the user; and
(b) estimating an impact of the at least one effect on the timed activities and/or the timed preferred user experiences.

Optionally, generating is based at least partially on (a) and (b). Optionally, generating is further based on additional data, including for example: previous consumption of the at least one active substance by the user; a user's medical history and condition; previous regimen(s) executed by the user; previous regimen(s) executed by other users (e.g. a population), and/or other data.

In some embodiments, the method comprises controlling a delivery device according to the delivery regimen, to deliver the amounts at the delivery times to a user.

In some embodiments, the method comprises locking the delivery device to prevent or limit delivery of the at least one active substance at one or more times defined according to the generated delivery regimen.

In some embodiments, the method comprises automatically issuing a notification regarding a current and/or future status of the at least one effect at one or more time points throughout the delivery regimen.

In some embodiments, the method comprises automatically recommending an activity or action based on the current and/or future status of the at least one effect.

In some embodiments, the method comprises storing the generated delivery regimen for future use.

In some embodiments, the method comprises storing the generated delivery regimen in correlation with at least one effect induced by the active substance and exhibited during executing of the delivery regimen.

In some embodiments, the method comprises storing the generated delivery regimen in correlation with feedback received regarding the delivery regimen.

In some embodiments, the feedback includes an indication of one or more of: how the user felt when executing the regimen, the level of the effect, the impact of the effect on the activities engaged by the user.

In some embodiments, the regimen comprises different time windows within which the effect on the user is within defined limits.

In some embodiments, the limits are defined according to a type of activity engaged by user, user preferences, and/or use restrictions.

In some embodiments, the method comprises accessing one or both of personal user data and population data, pertaining to pharmacokinetics and/or pharmacodynamics of the at least one active substance, and using the data in generating and/or adjusting the delivery regimen.

In some embodiments, estimating takes into account an expected lasting duration of the at least one effect.

In some embodiments, the at least one active substance induces or influences a clinical effect on the user.

In some embodiments, the at least one active substance induces or influences a psychoactive effect on the user.

In some embodiments, the at least one active substance comprises a cannabinoid.

In some embodiments, the preferred user experience includes a desired potency of an effect and/or a type of effect induced by the at least one active substance.

In some embodiments, the type of effect includes one or more of a clinical effect and non-clinical effect.

In some embodiments, the type of effect is from the group of analgesic effect, uplifting effect, sedative effect, calming effect, aphrodisiac effect, focus effect, social effect, appetizing effect, creative effect, energizing effect, and euphoric effect.

In some embodiments, the personal user data includes a medical condition of the user.

In some embodiments, the method comprises estimating an expected blood plasma level of the at least one active substance at one or more selected times during or following executing of the delivery regimen.

In some embodiments, the use restrictions include a maximal blood plasma level of the at least one active substance.

In some embodiments, the use restrictions include a minimal blood plasma level of the at least one active substance.

In some embodiments, the method comprises dynamically modifying the delivery regimen according to indications related to changes in a user's activity schedule, and/or according to sensed data indicative of a user status.

In some embodiments, the method comprises initially generating one or more alternative delivery regimens for implementing if and when a change in timed activities and/or a change in timed preferred user experiences takes place and/or a sensed effect on the user passes a predefined limitation.

In some embodiments, the method comprises estimating a likelihood of the user to experience a certain psychoactive state at one or more selected times during or following executing of the delivery regimen.

In some embodiments, the method comprises estimating a likelihood of the user to reach a certain plasma level of the active substance at one or more selected times during or following executing of the delivery regimen.

In some embodiments, the method comprises pairing the delivery times with the amounts, wherein the amounts vary.

In some embodiments, the regimen sets delivery of a placebo at one or more of the delivery times.

In some embodiments, the method comprises defining a maximal level of the at least one effect, wherein the delivery regimen is planned such that the at least one effect does not exceed the maximal level.

In some embodiments, the method comprises permitting delivery of placebo if the maximal level is reached and until delivery of additional active substance is permitted.

In some embodiments, delivery comprises pulmonary delivery via a metered dose inhaler device.

According to an aspect of some embodiments a system is provided for planned delivery of at least one active substance to a user, comprising:
a user interface configured to receive as input at least one of timed activities to be engaged by the user, timed preferred user experiences;
a controller programmed to generate a delivery regimen including at least one of delivery times and amounts of the at least one active substance, based at least partially on:
   (a) estimating at least one of a timing and magnitude of at least one effect expected to be induced by the at least one active substance on the user; and
   (b) estimating an impact of the at least one effect on the timed activities and/or the timed preferred user experiences;
and
a delivery device associated with the controller, the delivery device operative to deliver to the user the at least one active substance according to the delivery regimen.

In some embodiments, the delivery device is configured to be locked to thereby prevent or limit delivery of at the least one active substance at one or more times defined according to the generated delivery regimen.

In some embodiments, the controller is programmed to generate the delivery regimen based on one or both of personal user data and population data, pertaining to pharmacokinetics and/or pharmacodynamics of the at least one active substance.

In some embodiments, the controller is programmed to generate the delivery regimen based on use restrictions.

In some embodiments, the controller and the delivery device are in communication with a memory which stores delivery times during which the at least one active substance is delivered to the user via the delivery device.

In some embodiments, the memory stores one or more delivery regimens and effects induced by the at least one active substance on the user at one or more times during executing of each of the one or more delivery regimens.

In some embodiments, the controller comprises a communication module which is in communication with a user's personal digital device.

In some embodiments, the communication module receives and/or transfers data to a user's personal digital calendar.

In some embodiments, the controller is programmed to generate the delivery regimen according to one or more pharmacokinetic limits and/or one or more pharmacodynamic limits.

In some embodiments, the controller is programmed to calculate a cumulative effect of a plurality of delivery events on the user, and to plan at least delivery times and amounts of the at least one active substance according to the cumulative effect.

In some embodiments, the delivery device is a metered dose inhaler device.

In some embodiments, the delivery device contains or is structured to be in operable contact with a plurality of dose units comprising known amounts of the at least one active substance, and to actuate use of the dose units according to the delivery regimen in response to an indication from the controller.

In some embodiments, the dose units comprise botanical material.

In some embodiments, the botanical material comprises cannabis, and the at least one active substance comprises THC.

In some embodiments, different dose units comprise different amounts of the at least one active substance.

In some embodiments, the user interface receives and/or acquires feedback from the user during and/or following executing of the delivery regimen.

In some embodiments, the user interface comprises or is in communication with one or more sensors or devices suitable for assessing an effect of selected delivery events or of a full delivery regimen on the user.

In some embodiments, the user interface notifies a user regarding their current pharmacodynamic state and/or current pharmacokinetic state based on the time that passed from one or more previous delivery events.

In some embodiments, the user interface provides a user with an expected blood plasma level of the at least one active substance at one or more selected times during executing of the delivery regimen.

In some embodiments, the controller limits delivery of the at least one active substance by the delivery device according to the delivery regimen.

In some embodiments, the controller is programmed to lock the delivery device.

In some embodiments, the controller is programmed to lock the delivery device at one or more of in between delivery events, when a predefined limit of the at least one effect had been reached, or when a non-permitted user attempts to use the delivery device.

In some embodiments, the controller comprises a processor which calculates the expected interaction of one or both of the timed activities and the timed preferred user experiences with the at least one effect.

In some embodiments, the processor is programmed to calculate a tradeoff between a timed activity and a preferred user experience, and to define at least one of the delivery times and amounts according to the tradeoff.

In some embodiments, the controller is configured to notify a user, via the user interface, regarding a current and/or future status of the at least one effect at one or more time points throughout the delivery regimen.

According to an aspect of some embodiments a method is provided for generating a delivery regimen of at least one active substance to a user, comprising:
receiving input including a user's schedule and at least one preferred user experience during at least a part of the schedule;
automatically generating a delivery regimen including at least one of delivery times and amounts of the at least one active substance, wherein generating a delivery regimen is at least partially based on at least one effect induced by the at least one active substance on the user.

In some embodiments, generating takes into account an initiation time of the at least one effect, and a lasting duration of the at least one effect.

According to an aspect of some embodiments a method is provided for generating a delivery regimen of at least one active substance to a user via inhalation, comprising:
receiving input including one or both of timed activities to be engaged by the user, timed preferred user experiences; and
automatically generating a delivery regimen including at least one of administration times and amounts of the at least one active substance, based on an expected timeline of a blood plasma level of the at least one active substance and based on how the blood plasma level would impact one or both of the timed activities and the timed preferred user experiences.

According to an aspect of some embodiments a method is provided for generating time-based recommendations to a user, comprising:
receiving input including a timing and amount of one or more doses previously taken by the user and/or one or more planned doses; and
automatically generating time-based recommendations based at least partially on estimating at least one of a timing and magnitude of at least one effect expected to be induced by the at least one active substance on the user, the time-based recommendations including an indication of the user's capability for engaging in one or more activities, wherein the capability is correlated with the at least one effect; and
outputting the recommendations.

In some embodiments, the input is obtained by automatically accessing information on a delivery device used for delivering the at least one or more doses previously taken by the user.

In some embodiments, the input is received via a user interface.

In some embodiments, the recommendation is set for a pre-defined likelihood of the user being capable to engage an activity.

In some embodiments, the recommendation includes a probability of the user's capability to engage an activity at a certain time.

In some embodiments, the recommendation includes a regimen of future deliveries of the same active substance previously taken by the user.

In some embodiments, the recommendation includes a regimen of future deliveries of a different active substance which is known to affect at least one of the timing and magnitude of the effect of the active substance previously taken by the user.

In some embodiments, delivery is via inhalation.

In some embodiments, the method comprises controlling a delivery device to deliver the active substance according to the regimen.

In some embodiments, the method comprises locking the delivery device to prevent additional delivery until receiving an external indication.

In some embodiments, the active substance comprises a cannabinoid.

In some embodiments, the active substance is released by the system directly from raw cannabis.

According to an aspect of some embodiments a system is provided for generating time-based recommendations to a user, comprising:
a user interface configured to receive as input a timing and amount of one or more doses previously taken by the user and/or one or more planned doses;
a controller programmed to generate time-based recommendations based at least partially on estimating at least one of a timing and magnitude of at least one effect expected to be induced by the at least one active substance on the user, the time-based recommendations including an indication of the user's capability for engaging in one or more activities, wherein the capability is correlated with the at least one effect.

In some embodiments, the system comprises a delivery device configured to deliver the at least one active substance in accordance with the recommendations generated by the controller.

According to an aspect of some embodiments a method is provided for generating a delivery regimen of at least one active substance to a user, comprising:
receiving input including one or both of timed activities to be engaged by the user, timed preferred user experiences;
and
automatically generating a delivery regimen including at least one of delivery times and amounts of the at least one active substance, based at least partially on estimating at least one of a timing and magnitude of at least one effect expected to be induced by the at least one active substance on the user, and based on an expected interaction of the at least one effect with one or both of the timed activities and the timed preferred user experiences.

Unless otherwise defined, all technical and/or scientific terms used herein have the same meaning as commonly understood by one of ordinary skill in the art to which the disclosure pertains. Although methods and materials similar or equivalent to those described herein can be used in the practice or testing of embodiments, examples of methods and/or materials are described below. In case of conflict, the patent specification, including definitions, will control. In addition, the materials, methods, and examples are illustrative only and are not intended to be necessarily limiting.

Implementation of the method and/or system of embodiments can involve performing or completing selected tasks manually, automatically, or a combination thereof. Moreover, according to actual instrumentation and equipment of embodiments of the method and/or system, several selected tasks could be implemented by hardware, by software or by firmware or by a combination thereof using an operating system.

For example, hardware for performing selected tasks according to embodiments could be implemented as a chip or a circuit. As software, selected tasks according to embodiments could be implemented as a plurality of software instructions being executed by a computer using any suitable operating system. In some embodiments, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments are herein described, by way of example only, with reference to the accompanying drawings. With specific reference now to the drawings in detail, it is stressed that the particulars shown are by way of example and for purposes of illustrative discussion of embodiments. In this regard, the description taken with the drawings makes apparent to those skilled in the art how embodiments may be practiced.

In the drawings:

FIG. 1 is a flowchart of a general method for automatically generating a delivery regimen of at least one active substance to a user, according to some embodiments;

FIG. 2 is a diagram of a delivery planning and executing system, according to some embodiments;

FIG. 3 is flowchart of a method for automatically generating and implementing a delivery regimen of at least one active substance to a user, according to some embodiments;

FIGS. 4A-B are flowcharts of a method for providing a user with predicted effects of at least one active substance delivered according to a predefined regimen (4A); and a method for notifying a user regarding a blood plasma level of the at least one active substance (4B), according to some embodiments;

FIGS. 5A-B graphically present results of a clinical trial testing pharmacokinetic effects (5A) and pharmacodynamic effects (5B) of an active substance over time;

FIGS. 6A-B depict a schematic chart indicating a likelihood of reaching a certain blood plasma level of at least one active substance delivered to a user (6A); and a schematic graph presenting an expected cumulative effect of multiple deliveries of at least one active substance on the blood plasma level (6B), according to some embodiments;

FIGS. 7A-B graphically present schematic examples of delivery regimens planned according to a desired pharmacokinetic limit (7A) and a desired pharmacodynamic limit (7B), according to some embodiments;

FIG. 8 is a schematic example of a delivery regimen planned to achieve a predefined pattern of limits set according to user preferences, according to some embodiments;

FIG. 9 is a schematic example of a planned timeline including planned activities and a desired psychoactive state during those activities, and an actual timeline including a change in activity and the manner in which it affects the devilry regimen, according to some embodiments; and

FIGS. 10A-E are examples of user interface screens of active substance delivery planning application, according to some embodiments.

### DESCRIPTION OF SPECIFIC EMBODIMENTS

The present disclosure, in some embodiments thereof, relates to delivery of at least one active substance to a user, and, more particularly, but not exclusively, to systems and methods for automatically generating a delivery regimen of the at least one active substance to the user.

A broad aspect of some embodiments relates to generating a delivery regimen of at least one active substance to a user based on an expected interaction between a user's activities and/or preferred experience, and at least one effect induced by the active substance on the user. In some embodiments, the delivery regimen is generated according to the manners in which the at least one effect induced by the active substance impacts the current and/or planned activities by the user and/or impacts the current and/or planned preferred experience.

In some embodiments, the induced effect comprises a pharmacokinetic effect, for example, a blood plasma concentration profile of the active substance. In some embodiments, the induced effect comprises a pharmacodynamic effect, for example, a psychoactive effect.

An aspect of some embodiments relates to generating, based on input including a user's activity schedule and/or a user's preferred experience, a delivery regimen including at least a set of delivery times and/or doses to be delivered at the delivery times. In some embodiments, the input includes one or more of time of commencement of an effect, time of termination of an effect, a duration of an effect and/or a degree of the effect (e.g. on a virtual analog scale and/or a precise numeric value and/or by reference to one or measurable correlated properties).

In some embodiments, generating involves estimating for the one or more effects expected to be induced by the active substance, a potency of the effect, a lasting duration of the effect and/or a cumulative effect of multiple doses, and associating the estimation with the user's planned activities and/or the user's preferred experience, so as to provide the user with a regimen optimized for their specific needs. Optionally, planning of the regimen involves calculating when one or more effects are expected to be exhibited, the magnitude of such effects at one or more points in time and/or how long one or more effects are expected to last, and pairing these time periods with scheduled activities of the user.

In some embodiments, generating involves estimating how the effect induced by the active substance would impact the activities planned by the user. In some embodiments, generating involves estimating how the effect induced by the active substance would impact the desired experiences (e.g. mental states and/or physical states) of the user. Manners in which the effect may influence or impact the planned activities may include enhancing the user's performance during an activity (for example, help the user focus); facilitate or ease an activity (for example, calm the user down before sleep); additionally or alternatively, the effect may interfere with a planned activity (for example, make the user drowsy when they need to drive), and the delivery regimen is generated taking that into account. Optionally, the delivery regimen is planned to reduce or prevent the interference.

In some embodiments, generating involves accessing data, such as personal user data, population data, pharmacokinetics/pharmacodynamics data pertaining to the at least one active substance, and/or restriction related data. In some embodiments, the regimen is planned in accordance with the accessed data. In some embodiments, automatic generating of the delivery regimen includes or is performed by integrating the input with the accessed data, and mathematically calculating an optimized plan. Optionally, integrating includes a tradeoff between a scheduled activity (for example, the type of activity, the timing of the activity) and the delivery times and/or amounts of the active substance.

In some embodiments, generating involves defining one or more limits (e.g. thresholds and/or ranges), for example limits of the at least one effect induced by the active substance. Optionally, the limits are set for specific times or time periods. Optionally, the limits are set in correlation to the activity planned. Some examples of limits include: a maximal blood plasma level of the active substance; a minimal blood plasma level of the active substance; a defined psychoactive level (e.g. how drowsy or euphoric the user feels).

An aspect of some embodiments relates to generating recommendations to a user of at least one active substance, based on input related to one or more previous doses and/or planned doses of the active substance consumed by the user. In some embodiments, a timing of the dose and/or the dose size are provided as input, and based on at least one effect expected to be induced in the user by the active substance, recommendations regarding activities to be engaged by the user, future doses to be taken, activities to avoid and/or other recommendations are generated. Optionally, the recommendations are generated based on accessed data, such as data pertaining to an initiation time and/or lasting duration and/or magnitude of the effect, as previously exhibited by the same user and/or by a population of users. In an example, input regarding a previous dose consumed by the user is provided, and a recommendation regarding when the user shall be fit for engaging a certain activity (such as driving) is generated. In some embodiments, the recommendation includes an indication (optionally within a set likelihood or range thereof) of the user being capable to perform an activity.

In some embodiments, system and/or devices, for example as described herein, are configured to receive and/or access data related to the user, for example regarding previous doses taken by the user, and to output a recommendation according to the data. In an example, the system accesses and/or receives input regarding a dose of an active substance taken by the user. In an example, the system obtains input from a device that delivered the dose.

The user may then inquire the system, for example via the user interface (e.g. via a cell phone app) for recommendations such as: should I be able to drive now? Should I be able to drive in two hours? When should I be fit for driving? The recommendation generated by the system may take the form of one or more of: a yes/no answer; a likelihood of the user capability to perform an activity, which may be set as a percentage range (optionally, the likelihood is presented as a numeric indication; additionally or alternatively, a schematic visual indication such as a bar chart, use of icons, color, and the like is presented); indication of a time period that the user should wait and/or a specific hour by which the user should be capable of engaging an activity; an alarm and/or other notification, optionally including a safety warning (for example, the system receives input regarding a previous (e.g. large) dose taken, and then detects (e.g. via sensors, such as GPS sensor and/or acceleration sensor) that the user is at a velocity that might be indicative of driving—in which case the system may generate a question to the user to confirm that they are the passenger, advise to stop driving, and/or other alarm or notification to the user or another person.

An aspect of some embodiments relates to automatically adjusting a delivery regimen of at least one active substance to a user in real time, during executing of the delivery regimen. For example, a delivery regimen may last one or more hours (e.g. 2, 4, 5, 12 or 18 hours) and an adjustment may take place within such timeframes. In some embodiments, adjustment comprises re-setting one or more of delivery times and/or doses (e.g. re-defining amounts, types of substances, combining two or more substances, and/or other).

In some embodiments, adjustment of the regimen is due to: a change in a current or future scheduled activity; a change in user preference (e.g. the user desires a different type and/or strength of experience); a change in externally imposed restrictions (e.g. the user plans to drive soon, and there is a maximal limit to the blood plasma level desired by user or permitted by law); feedback from the user or a user's request; the current effect induced on the patient or a physical or psychoactive status of the user, for example as monitored by one or more sensors and/or provided as input by the user or by another; and/or other changes or indications.

In some embodiments, adjustment of the regimen is due to feedback obtained from a substance delivery device regarding a delivery event, including information regarding the precise time of delivery and/or the amount and/or type of substance(s) delivered. For example, in the event that a user takes a dose before or after a scheduled event and/or the actual dose delivered is slightly above or below the scheduled dose. This can at times compensate, for example, for expected variation of events and/or in the event of user mistake or device malfunction.

In some embodiments, user input includes at least one of several classes of user indications/information, including user semi-controlled (voluntarily or involuntarily) indication and user uncontrolled (involuntarily) indication. Receiving user feedback may include receiving user-controlled indication and/or information, wherein the user may provide input regarding the perceived and/or sensed effect and/or a desired range of effect. For example, inputting one or more responses to interrogation via the device's user interface and/or by a practitioner (e.g., grade a degree of pain and/or a degree to a psychoactive effect), while the user has control on the input he provides. A response may include grading on a scale (e.g., a scale of pain from 1 to 10), a Yes/No response, and/or a temporal description of an effect (“when did you notice that pain began to dissipate?”, “how did you feel during the time from inhalation to interrogation?” “since when are you feeling drowsy” etc.). The interrogation may be periodic (e.g., once a day or every several hours or upon inhalation or as a requirement before inhalation) and may be limited to only a part of the period of regimen. For example, interrogation may cover the initial 6, 12 or 24 hours in order to determine a regimen and then repeated periodically (e.g., once a day or once a week or once a month or upon desire) in order to confirm the regimen and/or readjust it.

Receiving user feedback may include receiving user semi-controlled indication, wherein the user may participate in measuring the user's status; while user compliance is needed for these indications, the user has little or no control on the result. For example, a user may be instructed to attach a sensor to his body that communicates with the device or allow sensing a property (e.g., eye redness). Optionally and alternatively, a user interface may test the user, for example by following pupils and/or instructing the user to perform a task. Examples for tasks may include, without limitation, following a mark on a screen with your eyes; dragging a mark on a screen through a pattern without touching walls; completing mental tasks (such as solving mathematical equations or answering questions); testing memory by games; and testing concentration.

Receiving feedback may include receiving user uncontrolled indication, wherein the inhaler or a device associated with the inhaler may sense one or more properties of a user as an indication of the user's status and use that as an indication of one or more effects of the inhaled dose regimen on the user. For example, sensing mouth temperature during inhalation and/or pupil size without prompting the user, each of which may indicate pharmacodynamic effect and thus serve as a pharmacodynamic effect; sensing tremor or a variation in tremor; and sensing heart rate and/or blood pressure, for example by interaction with a sensor worn by the user and/or implanted in the user.

In some embodiments, the initial delivery regimen is generated under the assumption that one or more changes are expected to occur, optionally requiring an adjustment of the regimen. Optionally, the delivery regimen is planned with one or more “fallback” or alternative regimens for when and if a change takes place, for example a change in a scheduled activity.

In some embodiments, the regimen is initially defined to include one or more pre-set times for requesting and/or obtaining input from the user, based on which adjustments of the upcoming regimen may be made. For example, a system (e.g. a system controller, for example as described herein) may be programmed to request, at one or more pre-set times during executing of the regimen, feedback from the user, for example regarding their current state, and modify the initial regimen accordingly. Optionally, input is obtained via one or more sensors, using the sensed information to assess a current state of the user and modifying the initial regimen accordingly. In an example, the system is pre-programmed to obtain or request input from the user right before one activity ends and another one is expected to begin, and to modify the regimen based on how the user is feeling and/or based on whether the upcoming activity has changed from an activity that was initially planned.

An aspect of some embodiments relates to a system for automatically generating a delivery regimen of at least one active substance, and delivering the active substance to a user via a delivery device controlled according to the regimen. In some embodiments, the system comprises a controller which controls operation of the delivery device, the controller comprising or is in communication with a processor which calculates the delivery regimen (for example, calculates delivery times and doses to be delivered) based on input received and/or inserted to the system. In some embodiments, system input is obtained or received via a user interface (from the user of the delivery device or another), optionally configured on a personal electronic device, such as a cell phone. In some embodiments, the system comprises a communication module, programmed to communicate with one or more of the user interface, such as for communicating a generated regimen to a person (e.g. the user) or device and/or receiving input from a person (e.g. the user, a caregiver, a friend and/or a physician, etc.); a remote server and/or a memory component, including data such as one or more saved delivery regimens (and optionally their associated effects) for the individual user and/or for a population of users.

In some embodiments, the controller is programmed to control operation of the delivery device according to one or more pre-defined limits. In an example, if a limit is reached (such as a maximal blood plasma level, a selected psychoactive state), the controller instructs the delivery device to deliver or permit delivery of a placebo. In another example, if a limit is reached, the controller instructs locking of the delivery device, optionally until another dose is permitted. In yet another example, the controller instructs locking of the delivery device during the regimen, and unlocking it as required in compliance with regimen constraints.

In some embodiments, the system is configured issue a notification (e.g. to the user), for example via the user interface, regarding a current and/or future status of one or more effects induced by the active substance. Optionally, the system provides recommendations to a user, which may relate to either an activity engaged by the user, or to use of the delivery device. For example, the system may recommend a user to wait a certain time period before an activity (e.g. driving or doing important work or performing a difficult task). In another example, the system may recommend to a user regarding how to enhance or reduce an effect, such as a psychoactive effect, e.g. by suggesting the user to drink a glass of wine or water or juice or inhale an additional substance having a desired effect following a dose of the active substance.

In some embodiments, the system is configured to set a maximal dose, optionally defined according to various constraints. Optionally, based on feedback sensed and/or inputted by the user, the system adaptively learns what is the optimized maximal and/or minimal dose per the user and/or per the situation. For example, the user inputs instructions that they plan to drive in 1 hour. The system sets the input as a constraint and defines a maximal dose. At 1 hour following taking that dose, the user still feels an effect, for example, still feels “high”. The system learns from that effect, and may set future maximal doses to a lower level or associate a longer period between the same dose and activities such as driving. In another example, the user wants to feel “high” at 30 minutes post-delivery, but the maximal dose provided was not sufficient to induce that effect—the system learns and sets a higher maximal dose for future regimens. Optionally, insights derived from AI and/or machine learning, based on user data and/or on data from a plurality of users or user groups, and/or other factors is used to generate the initial regimen and/or adjust it.

In some embodiments, the delivery device is a metered-dose inhaler. Optionally, the inhaler comprises or is configured to receive or attach to a plurality of dose units, for example contained in a cartridge or magazine. In some embodiments, the dose units (and/or their content) are arranged and/or selected for use by the device according to the delivery regimen.

Systems and/or devices (e.g. inhaler devices, dose units, cartridges) for example as described in: PCT Publication WO2012/085919; PCT Publication WO2016/001925; PCT Publication WO2016/001926; PCT Publication WO2016/001921; PCT Publication WO2017/122201; and/or PCT Publication WO2017/122196 are incorporated herein by reference.

An aspect of some embodiments relates to predicting, within a predefined likelihood, a blood plasma level of at least one active substance at one or more times following delivery of the active substance to the user. For example, the predefined likelihood may be 50% or more, 66% or more, 75% or more, 80% or more, 90% or more, 95% or more, or 99% or more. In some embodiments, the prediction takes into account one or more of personal user parameters, use history of the user (e.g. a previous single delivery event and/or a previous executed regimens), and a pharmacodynamic profile associated with therewith, population-based statistics, clinical research results, regulatory restrictions (e.g. maximal permitted blood level for performing a regulated task) etc.

In some embodiments, a system for example as described herein, is configured to issue a notification (in advance and/or in real time) regarding a user's current or expected blood plasma level of the active substance.

An aspect of some embodiments relates to actively controlling a user's mood and/or psychoactive state by planning delivery of at least one active substance and/or by planning and/or suggesting one or more user actions. In some embodiments, the system (for example as described herein) generates a regimen which suggests or recommends to the user when to act. The suggestions may be related to using the delivery device (e.g. when to use to the device), and/or to performing other actions which may affect the user's psychoactive state or mood, optionally when performed in conjunction with use of the delivery device. The suggested actions may be determined according to predefined input (e.g. as known or suggested for a general population), input provided by the user and/or based on previous effects one or more specific actions had on the user. Examples for suggested actions may include: listening to music (in the event that music, optionally of certain type, is known to and/or has shown to affect users (in general) and/or affect the specific user); performing physical exercise (for example in the event that physical exercise is known to or has to affect the specific user); dancing; drinking, eating, sleeping, changing environmental conditions such as by changing lighting; using drugs (e.g. alcohol, hallucinogenic drugs, such as LSD and/or psilocybin); and/or using (e.g. by consuming, smelling) other substances which may act as mood stabilizers and/or muscle relaxants and/or assist in treating nausea and/or anxiety, for example Lavender species, such as L. dentata, L. angustifolia, L. latifoliate, and L. hybrida; Valeriana officinalis; and/or Mentha piperita.

The suggested action may have its own independent effect on the user and/or may have a synergistic or antagonistic effect with one or more other actions or substances, such as a synergistic effect with an active substance delivered by the delivery device. For example, THC is delivered via the delivery device, and the generated regimen recommends the user to then listen to their favorite music to potentially enhance the psychoactive effect of the THC or to perform a physical task so as to reduce an effect of the THC dose.

In some embodiments, the regimen and/or or recommendations are generated taking into the account the user preferences and/or scheduled activities. For example, timing is calculated to ensure the user is capable of performing specific tasks when preferred or needed (e.g. when driving), and/or assisted into a desired psychoactive state or mood when such mood is intended.

It is noted that systems and/or method for example as defined herein may be associated with medical (e.g. therapeutic) use and/or with non-medical (e.g. recreational) use. In some embodiments, when used for medical purposes, generating the delivery regimen takes into account personal and/or general health restrictions. Optionally, for therapeutic needs, delivery is controlled so that even if non-desired effects are present (e.g. a psychoactive effect, if not desired), a user may be provided with additional active substance (e.g. at certain selected times, or per user preference) to improve the user's condition for even a limited, selected time period. Optionally, a regimen may be associated with both medical and non-medical use, such as a patient using medical cannabis, option to also use the plant recreationally. This may include maintaining a minimal therapeutic effect and controlling recreational effects during a night out.

As referred to herein, the term “experience” (also mentioned as “user preferred experience”; “preferred experience”; “desired experience”) is intended to cover both medical and non-medical purposes, depending on the type of use being made and/or the user's needs and/or the type of active substance delivered. The preference may be set by the user herself or by another on behalf of the user (e.g. a friend for recreational purposes, a caregiver or physician for medical purposes, etc.). For example, a recreational preferred experiences may include a psychoactive level (e.g. feeling “high”, euphoric or drowsy); a therapeutic preferred experience may include alleviating a symptom (for example pain or nausea). Optionally a desired experience includes a lack of a given effect, such as not reaching a drowsy effect, even regardless of other requests.

In some embodiments, the delivered substance is an active substance. In some embodiments, the substance is released from a source material by means of heating and/or airflow.

In some embodiments, the active substance affects an effect of one or more additional substances delivered to the user. Such additional substances may be delivered before, in conjunction and/or following delivery of the active substance. Optionally, the one or more additional substances are delivered via the same delivery device being used to deliver the active substance (e.g. an inhaler device). Additionally or alternatively, the additional substances are delivered and/or consumed and/or administered in a manner different than a manner in which the active substance is delivered. In some embodiments, when multiple devices are used, delivery is coordinated by a processor in association with one or more of the devices, and/or in communication with the user via a user interface.

The term “active substance”, as referred to herein, can include a substance that comprises one or more compounds having at least one medicinal and/or somatic and/or sensory and/or psychotropic effect on a user. In some embodiments, more than one active substance is provided. In some embodiments, a plurality of active substances are used, optionally simultaneously. In some embodiments, the plurality of different substances differ from each other, for example differ in substance type and/or in concentration and/or content of compounds.

A sensory or psychotropic effect, in accordance with some embodiments, optionally corresponds to an effect that can be perceived by the user (e.g. a patient). It is noted that in some cases a psychotropic effect may not be accurately perceived by the user. In some embodiments the effect is perceived on hindsight, after it had been terminated. Examples of psychotropic effect include, without limitation, paranoia, anxiety, panic attack, euphoria, pseudo-hallucinatory, sedation, conscious perception variation, joviality, metacognition and introspection, an enhanced recollection (episodic memory), amnesia, a sensuality variation, a variation in awareness of sensation and a variation in libido, dizziness, ataxia, euphoria, perceptual alterations, temporal distortion, intensification of ordinary sensory experiences, short term memory, and attention, impaired reaction, skilled activity, verbal fluency, dependence, melancholy and depression.

A somatic effect, in accordance with some embodiments, optionally corresponds to an effect or a symptom which can be perceived by the user or otherwise determined or measured. Examples for somatic symptoms include, without limitation, pain, migraine, nausea, dry mouth and a sensation of cold or hot hands and feet, increased heart rate, increased cerebral blood flow (e.g., migraine symptoms, “head pressure”), dilation of bronchial passages (e.g., coughing and difficulty breathing), dilation of blood vessels (e.g., shivers, skin redness, blushing), eye redness and pupil dilation, dry mouth, thirst, hunger or food craving. The terms “timed activities”, “user schedule”, “planned activities”, “activity schedule” or the like, as used herein, can cover regular (e.g. daily) and/or irregular activities performed by the user, and the time during which the activity is performed or engaged by the user.

In some embodiments, the timing is an absolute value (e.g. at 14:00-15:00 the user plans to drive). In some embodiments, the timing is relative timing, for example with respect to other activities (e.g. “in two hours” or “after work”, the user plans to drive). Timing may pertain to one or more points along a timeline or schedule. For example, a single point in schedule is referred to (e.g. the user indicates that at 18:00 they will start driving), or, for example, more than one point in schedule is referred to (e.g. the user indicates that within the next 30 minutes they will drive).

In some embodiments, timing pertains to the end of an activity (e.g. the user is at a party and plans to leave in 3 hours, and prefers to feel “high” during the party, but not after). In some embodiments, timing pertains to initiation of an activity (e.g. in 3 hours, the user will start driving). In some embodiments, timing pertains to initiation and ending of an activity (e.g. the user plans to attend a party for 3 hours, and wants to feel “high” as soon as the party starts, but be in full mental capacity at the end of the 3 hours).

In some embodiments, timing of activities overlaps (e.g. the user eats when attending a party). Such timing considerations, for example, may be taken into account when generating the delivery regimen.

Before explaining at least one embodiment in detail, it is to be understood that this disclosure is not necessarily limited in its application to the details of construction and the arrangement of the components and/or methods set forth in the following description and/or illustrated in the drawings and/or the Examples. Other embodiments are possible, and some embodiments can be practiced or carried out in various ways.

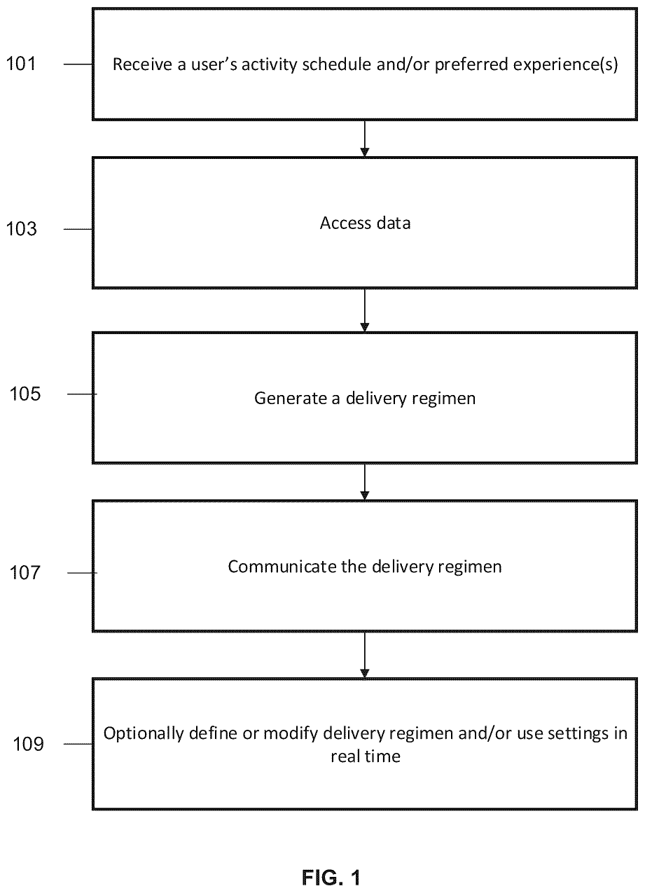

Referring now to the drawings, FIG. 1 is a flowchart of a general method for automatically generating a delivery regimen of at least one active substance to a user, according to some embodiments.

In some embodiments, a delivery planning system receives one or both of a user's activity schedule, a user's preferred experience (101).

In some embodiments, a user's activity schedule includes the user's plan for an upcoming time period (e.g. the next few hours; the following day), including, for example, daily activities such as eating, driving, exercising, engaging in a social activity, sleeping, performing mental tasks (e.g. at work), performing physical tasks (e.g. that require agility, stability, force and/or dexterity) taking a test, performing a dangerous task, engaging in competitive sports, performing medical tests, taking medications (that may affect or be affected by the regimen), going on vacation.

In some embodiments, a user (or another on his/her behalf) inserts the user's schedule as input; additionally or alternatively, the system is automatically updated with the schedule, for example by accessing a user's calendar (for example via a calendar app on a user's personal device, such as a cell phone).

In some embodiments, the preferred experience is entered as input. Optionally, the input is provided in response to an inquiry by the system (e.g. via a system user interface). In some embodiments, a preferred experience is defined for different activities and/or different time windows in the user's schedule. Optionally, one or more time-limits are set for an inserted preferred experience, such as a start time and an end time, by which the at least one effect of the active substance is no longer sensed or exhibited.

In some embodiments, the preferred experience includes a desired mood, a desired mental state, and/or a desired psychoactive effect. Various examples of experiences include analgesic effect, uplifting effect, sedative effect, calming effect, aphrodisiac effect, focusing effect, social effect, appetizing effect, creative effect, energizing effect, euphoric effect, and/or other effect. In some embodiments, the preferred experience includes a desired physical state, a desired dexterity, a desired ability to apply force, a desired stability, a desired agility and/or any other physical state. In some embodiments, a user selects a desired potency of an effect, for example: strong, moderate or weak or a more precise degree (e.g. on a 1-10 visual analog scale). Optionally, one or more effects are given more weight than others. For example—a regimen may include a fixed threshold (e.g. no psychoactive effect and/or no physical impairment permitted by a given time or during a defined period), or a given blood concentration may not be exceeded at any given time, regardless of other parameters.

In some embodiments, a preferred experience is selected in accordance with the type of activity engaged by the user. In some embodiments, the user defines the experience; additionally or alternatively, the system automatically selects a preferred experience based on the type of activity engaged by the user.

Some examples of input in which a preferred experience is associated with an activity engaged by the user may include requesting a calming effect before sleeping; requesting an appetizing effect during eating; requesting to avoid an appetizing effect during eating (e.g. for weight loss), requesting an energizing effect during exercise; requesting a creative effect during work; requesting an uplifting effect when going to a bar; and/or other.

In some embodiments, the system accesses data, including one or more of personal data, population data and restriction-related data (103). For example, one or more of the personal and population data may include prior or current pharmacokinetics and/or pharmacodynamics data.

Personal data may include, for example, the user's age, weight, sex, medical condition, BMI, routine activities, geographical location, profession, marital status, recent use, sensed physical properties (e.g. heart rate, blood pressure, eye redness, stability, agility, etc.), sensed emotional properties (e.g. mapping of vocal emotion or facial expression) and/or other data.

Population data may include, for example, typical dosages of the active substance which are associated the population at large or with certain sectors of the population. Optionally, the sectors are defined in accordance with the personal data (for example, the system accesses data relevant to users of the same age range, sex, the same geographical location, and/or other).

Restriction-related data may include, for example: regulatory restrictions, such as a maximal blood plasma level of the active substance which is allowed while driving; medical restrictions, for example, restrictions for safe use in a user having a medical condition or risk, such as diabetes or cardiovascular issues; age-related restrictions (e.g. a maximal level allowed for a certain age range; preventing use in an under aged user), possible interaction with medication used by the same user (optionally including a regimen for use of the medication); and/or other restrictions.

Pharmacokinetics and/or pharmacodynamics data may include, for example, one or more pharmacokinetic and/or pharmacodynamic effects of the active substance, as known from literature, from a population database (at large or by sector), from previous uses by the same user, and/or other sources. In some embodiments, the effect is associated directly with an expected timeline of blood plasma level of the active substance. Optionally, the expected timeline of blood plasma level is calculated for the specific user based on literature data (e.g. data obtained during clinical trials), population data, previous use, and/or personal data.

In some embodiments, the system automatically generates, based on one or more of the activity schedule, preferred experience and/or accessed data, a delivery regimen for delivering the at least one active substance to the user (105). In some embodiments, the delivery regimen includes a timeline for administration of a plurality of doses. Optionally, the doses vary in amount of the active substance and/or the precise composition of the active substance formulation. For example, the doses may be provided in one or more reservoirs, one or more tapes and/or distinct dose units. Optionally delivered doses may be identical or may have different active substance(s) or different amounts of the substance(s), and/or having a different composition (such as the same active substance but with a different entourage).

In some embodiments, a plurality of different active substances are delivered, and the delivery regimen is generated based on at least one effect selected between: an additive effect, a synergistic effect and a counteractive effect between at least two of the active substances.

In some embodiments, the delivery regimen includes instructions according to which a delivery device (e.g. an inhaler) is programmed to deliver the active substance. In some embodiments, the delivery regimen includes instructions to be actively carried out by the user or by a caregiver, for example, to take/deliver a dose at a certain time, such as at a defined hour and/or at a defined time period before, during or after an activity. In some embodiments, the delivery regimen defines a maximal dose (i.e. amount of active substance) deliverable within a certain time limit, optionally permitting the delivery to take place in smaller equal or unequal batches. In such event, the regimen may be adjusted according to the time and amount of active substance in each taken batch.

In some embodiments, the delivery regimen generated by the system is communicated (107) to the delivery device, the user, and/or or a caregiver. The delivery regimen may be communicated as a set of instructions, for example including a set of timings for delivering the active substance from the delivery device. Optionally, the instructions further include operational settings such as which type of source material(s) should be used, when to renew the source material (e.g. replace a source material cartridge). Optionally, the instructions further include a planned interaction with the user, for example, when to obtain feedback from the user. In some embodiments, the instructions are timed based on one or more of: scheduled activities, doses scheduled to be delivered, a lasting duration of at least one effect induced in the user by the active substance, previously administered doses, restrictions, predictive analytics (including for example insights derived from AI and/or machine learning, based on user data and/or on data from a plurality of users or user groups, and/or other factors.

In some embodiments, during executing of the delivery regimen (and/or during the actual time period the regimen was planned for), the delivery regimen and/or use settings may be defined or modified. Optionally, the delivery regimen and/or use settings are set or adjusted in response to input (e.g. user feedback), received and/or collected at one or more time points during the planned time period. Optionally, the system interrogates the user regarding their experience (current status and/or past status within the same regimen), and adjusts the regimen accordingly. Optionally, the system detects changes in the activity schedule, and adjusts the regimen accordingly. Optionally the system obtains sensor data indicative of an effect of the user and/or activity of the user and adjusts the regimen accordingly and/or issues a notification (to the user, a caregiver or another). In some embodiments, use settings such as timing of delivery, dose size, device locking and/or unlocking, type of substance being delivered, and/or other use settings are set or adjusted. For example, if a user takes their maximal dose allowed per time, the delivery device automatically locks to prevent additional use. Optionally, the device automatically unlocks when a next dose is permitted.

In some embodiments, inactivating the device (for example when locking the device for use and/or locking the device to prevent use by users other than designated) may be carried out mechanically and/or via software control. Some examples for device inactivation may include obstructing airflow through the device, blocking one or more device conduits (e.g. by a valve); preventing use of a dose unit (e.g. preventing pulling out of a dose unit from a cartridge); affecting the electrical circuitry, not activating heating of the source material, and/or other. In some embodiments, software based device locking comprises setting the controller with a timer to prevent use during a selected time period, instructing to deliver a placebo in replacement of the active substance, requiring a code or other specified access (such as to prevent use by non-authorized users, e.g. a child) and/or other.

In another example, if a user takes their maximal dose allowed per time, placebo doses are delivered. In another example, the device automatically turns on when a dose is permitted (and/or off, which no doses are permitted or once a dose was taken).

In some embodiments, a lock can be overridden. In an example, the system interrogates the user, for example by having the user confirm an intent to change a locked setting. Other examples include requiring a password or code (which may be restricted to the user, a caregiver and/or a physician) and/or otherwise.

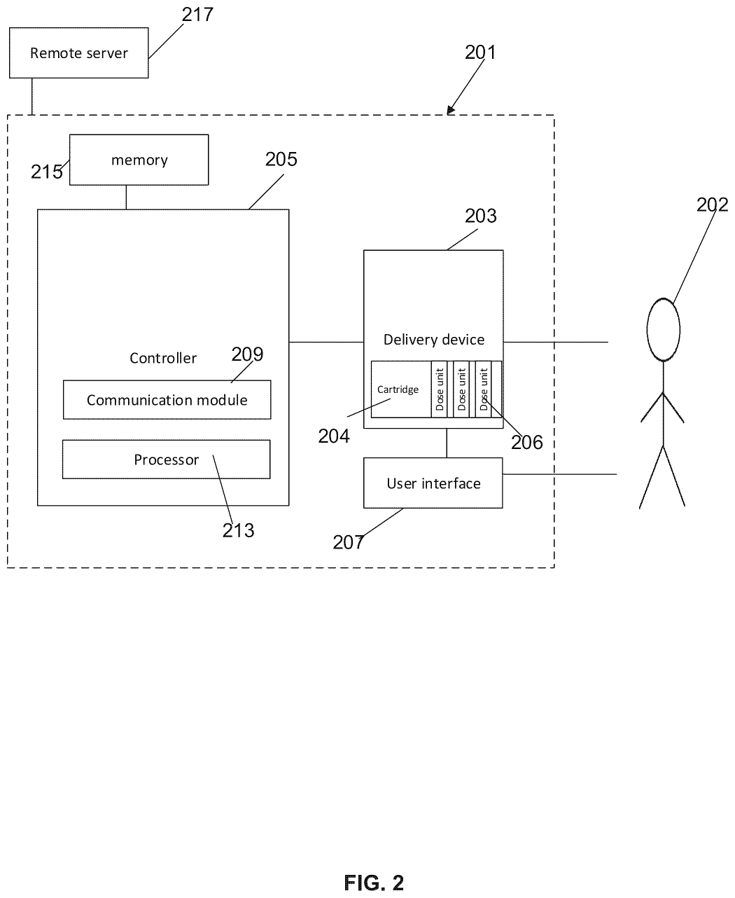

FIG. 2 is a diagram of a delivery planning and executing system, according to some embodiments.

In some embodiments, system 201 comprises a delivery device 203 for delivering at least one active substance to a user 202. In some embodiments, the delivery device is configured for releasing the at least one active substance from at least one source material. In an example, the source material comprises botanical material (e.g. plant material) and/or comprises a botanical or synthetic carrier to which the active substance was added.

In some embodiments, the delivery device is a medical metered-dose delivery device. Alternatively, the delivery device is non-medical, for example a recreational metered-dose device. As used herein, the term “metered dose” may mean that a delivered dose is within ±35% of a defined dose, within ±25% of a defined dose, or within ±15% of a defined dose, or intermediate, higher or lower percentage of a defined dose. For example, when a 1,000 μg dose is to be delivered, a ±15% range means that the actual dose may be between 850-1150 μg.

Optionally, the delivery device is an inhaler device, configured for pulmonary delivery of the at least one active substance to a user. Other delivery devices may include, but not limited to, devices for oral administration, on-skin (transdermal) administration, intravascular therapy, nasal administration, injection, parenteral administration, subcutaneous administration, intradermal administration, intramuscular administration, intravenous administration, intramedullary administration, intra-arterial administration, intrathecal administration, topical administration, rectal administration, mucosal administration.

In some embodiments, delivery device 203 comprises, or is structured to receive or to be in operable contact with, or to be attached to, a cartridge 204 (or other storage) containing a plurality of dose units 206. In some embodiments, a dose unit 206 houses the active substance or a source material from which the active substance is released.

In some embodiments, delivery device 203 releases the at least one active substance from a material. In an example, the delivery device comprises a heating mechanism for heating a source material, e.g. cannabis, in order to release one or more substances by vaporization, such as Δ9-tetrahydrocannabinol (THC), cannabidiol (CBD), and/or other cannabinoids and/or terpenoids.

Additionally or alternatively, delivery device 203 is configured to release the at least one active substance by spraying, aerosolizing, releasing from a capsule, and/or other methods for dispensing the active substance. In some embodiments, the delivery device dispenses a liquid and/or a powder. In some embodiments, delivery device 203 is controlled by a controller 205. In some embodiments, controller receives input, optionally via user interface 207. The input may include, for example, preferred user experiences, an activity schedule, and/or feedback, e.g. regarding a current or previous effect experienced by the user.

In some embodiments, based on the input data and/or accessed data (e.g. restriction related data, pharmacokinetic/pharmacodynamic data, population data) a processor 213 (e.g. a chip and/or other computational component) associated with controller 205, calculates a delivery regimen. In some embodiments, controller 205 sends operating instructions to delivery device 203 according to the generated delivery regimen. Additionally, or alternatively, the generated delivery regimen is communicated (e.g. via communication module 209 associated with controller 205) to the user or another (e.g. a caregiver). Optionally, communication is wireless.

In some embodiments, system 201 comprises a memory 215, for storing data such as personal user data, generated delivery regimens, feedback received during executing of the regimens, use data (e.g. record use events) and/or other data. In some embodiments, memory 215 is a component of the delivery device 203 itself. Optionally, data is recorded and/or stored locally.

In some embodiments, system 201 is in communication with a remote server 217, for example a cloud-based server. Optionally, the remote server stores population data, clinical data, restriction related data, information regarding different source materials and/or active substances, and/or other information.

In some embodiments, the controller is incorporated within the delivery device; alternatively, the controller controls the delivery device remotely.

In some embodiments, the processor is an independent component, functioning separately from the controller.

In some embodiments, the user interface is configured as a component of the delivery device, optionally including a screen (e.g. touch screen) and/or push buttons. Additionally, or alternatively, the user interface is configured as a dedicated application on a user's personal electronic device, such as a cell phone, a tablet computer, a smart watch, and/or other devices and/or at a physician's office.

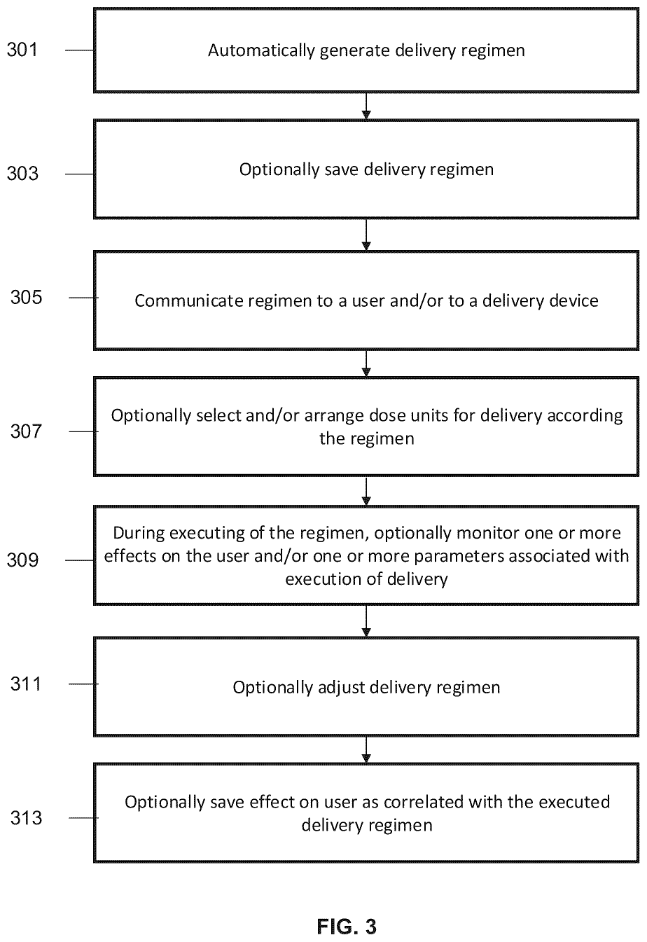

In some embodiments, computational and/or memory components for planning and/or communicating the delivery regimen are configured as a part of a cell phone, optionally as a designated cell phone app. In some embodiments, cell phone hardware components and/or sensors are used for obtaining user related data and/or data pertaining to the effects of treatment on the user. For example, voice recording via the cell phone microphone, GPS location assessment, motion sensing via a gyroscope and/or accelerometer of the cell phone, images and/or videos captured by the cell phone camera, and/or other. FIG. 3 is flowchart of a method for automatically generating and executing a delivery regimen of at least one active substance to a user, according to some embodiments.

In some embodiments, a delivery regimen is automatically generated (301), for example as described herein. Optionally, the regimen is saved (303), e.g. to a memory and/or a remote server. In some embodiments, the delivery regimen is communicated to a user and/or to a delivery device (such as to the device controller) (305).

Optionally, one or more dose units are selected and/or arranged for delivery according to the planned regimen (307). Specific dose units may be picked from a storage (e.g. cartridge) and optionally put in an order in which they are planned to be delivered. In some embodiments, selection and/or arrangement is carried out automatically, for example by a dedicated mechanism of the delivery device, such as a pulling mechanism which places the dose unit in a use-position for release of the active substance. Additionally, or alternatively, manual selection and/or arrangement is performed. Optionally the specific dose is extracted from a dose unit holding a greater amount of active substance by the delivery device mechanism. Optionally, in a vaporizing metered dose inhaler, the dose may be extracted by application of heat and/or controlling the airflow.

In some embodiments, during execution of the planned regimen parameters of delivery execution are monitored (309). The monitored parameters may include functional parameters relating to delivery execution which may influence the degree of adherence to the planned regimen and/or which may influence the timing, type and/or magnitude of an effect on the user. Examples for such delivery execution parameters may include one or more of the precise time(s) of delivery, the active substance(s) delivered, and/or operational features of the delivery device that may affect the delivery. In some embodiments, monitoring includes feedback received by sensor readings within the delivery device. In some embodiments, monitoring includes feedback received via a user interface. This may provide an estimation of one or more effects induced on the user by the active substance, but which might deviate from the planned regimen and may warrant adjustment thereof.

In some embodiments, during execution of the planned regimen properties of the user are monitored (309). This may provide detection of one or more effects induced on the user by the active substance or one or more effects due to some external cause, but which might affect the user's response/wellbeing during the regimen.

In some embodiments, monitoring involves collecting feedback from the user. Optionally, feedback is obtained by directly questioning the user, for example via the user interface, regarding their current experience and/or a desired future experience (e.g. in an upcoming time period, for example in the next one hour, two hours, 4 hours, 7 hours or intermediate, longer or shorter time periods.) Additionally, or alternatively, feedback is obtained indirectly, for example by having the user perform a task (e.g. play a game on the user interface) to test the user's current state, such as the psychoactive state.

In some embodiments, monitoring is carried out via one or more invasive or non-invasive sensors. Optionally, data collected by the sensors is indicative of pharmacodynamic effects. Optionally, data collected by the sensors comprises physiological feedback.

Examples of sensors may include: a touch screen, may be used for example to assess dexterity, eye-hand coordination, and/or a memory and cognition state; a gyroscope, accelerometer, proximity sensor and/or gesture sensor such as IR sensor may be used, for example, to assess motor skills; a camera and/or light source may be used, for example, to detect visual tracking, saccade variance, eye vascular expansion, pupil dilation and/or pulsation; an RGB illumination may be used, for example, to assess environmental perception; a magnetometer and/or GPS may be used, for example, to assess orientation; a speaker and/or microphone may be used, for example, to assess auditory and/or vocal skills; a temperature and/or humidity sensor may be used, for example, to assess a body temperature.

In some embodiments, one or more sensors are used for collecting data other than pharmacokinetic/pharmacodynamic related data, for example: a GPS sensor and/or a motion sensor may be used for assessing a current location and/or velocity of the user (or even activity, e.g. driving, walking), a camera may be used for assessing surroundings of the user (for example, if the user is among people; if the user is outdoors; etc.); performing emotional face recognition from images obtained by a camera; referring to the social feed of a user; referring to the user's online searches and activity; and/or other.

In some embodiments, the sensors are configured as a part of the personal electronic device, e.g. cell phone, and/or of the delivery device itself.

In some embodiments, the delivery regimen is adjusted (311). Optionally, adjustment is performed in response to one or more of feedback from the user, input by a caregiver or physician; data collected by sensors (optionally pertaining to a monitored effect), a change in the activity schedule, and a user request.

Some examples of adjustments may include:
a user provides feedback of a strongly sensed psychoactive effect, and asks to reduce the effect; in response, the system automatically reduces the next dose and/or delays it for longer.
an activity schedule changes, and the user needs to engage in an activity which involves a legal restriction on the blood plasma level of the active substance, such as driving. In response, the system notifies the user when their blood plasma level is expected to be low enough to drive, and/or modifies (optionally prevents) the next dose accordingly.
a location of the user is assessed (for example via GPS), indicating that the user is on their way to work. In response, the system automatically sets the next dose as one that would improve focusing. For example, a regimen may include a definition of a default home regimen (relatively high sedation and high symptom relief) and a default work regimen (relatively high focus at the expense of some symptom relief). A generated regimen may adjust automatically in real time based the default regimens and location changes.
a user indicates that they are suffering from a medical problem or a sickness (which may be a permanent condition or a temporary condition), and in response, the system automatically sets the next dose (changing a size of the dose and/or a type of active substance delivered and/or a timing of the dose) to treat the symptoms.
During a recreational regimen, a severe intoxication effect is sensed (e.g. based on excessive groggy motions and instability). This may lead the system to adjust the regimen by preventing the delivery of additional recreational doses, suggesting counter action (by the user, a caregiver and/or by delivering a counteracting substance) and/or issuing an alert to a predefined person.
A user wants to enhance abilities such as memory, focus, stamina, alertness and/or others, for example by using medicinal plants.

In some embodiments, the induced effect(s) are on the user, as correlated with the actual delivery regimen that was executed, are recorded and optionally saved (313). In some embodiments, these recordings are used as basis for future planning of a delivery regimen of the same user and/or of a population of users.

Optionally, in some embodiments, the user selects to repeat a saved delivery regimen.

Optionally, in some embodiments, the user (and/or the system, automatically) shares the saved delivery regimen with one or more other users, who uses a similar metered dose inhaler and may desire the same regimen. The user may also share with one or more other users an effect or experience induced by a delivery regimen. A potential advantage of sharing an effect or experience with one or more other users may include that the user does not need to physically provide the other user with a certain substance (e.g. botanical, for example a certain strain of cannabis) but rather only transfer digital data indicating the delivery regimen which the other user can use for experiencing a similar or like effect.

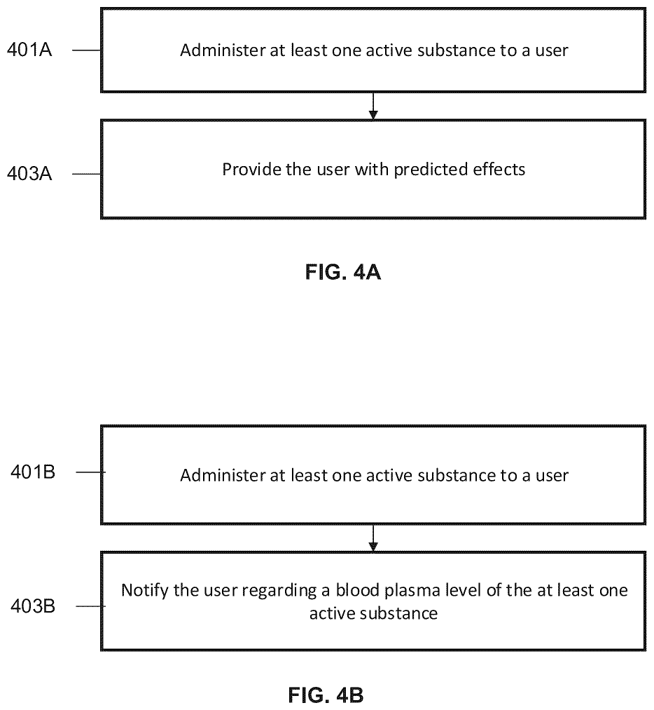

FIGS. 4A-B are flowcharts of a method for providing a user with predicted effects of at least one active substance delivered according to a predefined regimen (4A); and a method for notifying a user regarding a blood plasma level of the at least one active substance and/or provide an indication associated with the blood plasma level (4B), according to some embodiments.

In some embodiments, a user is administered the at least one active substance (401A). Optionally, the active substance is delivered according to the planned delivery regimen. Alternatively, the active substance is delivered separately from when the planned delivery regimen starts, for example, the user randomly decides to take a dose.

In some embodiments, the system (for example the system processor) calculates one or more expected effects induced by the active substance, for example at one or more selected time points during or following executing of the delivery regimen. Optionally, the effects are estimated based on one or more of clinical data, personal data, population data, or a combination thereof. Optionally, the system (e.g. processor) calculates an expected lasting duration for each of the effects, and/or generates a scale of degradation of the effect over time. In an example, the system predicts that a dose delivered at 8:00 AM will have no lasting effect after 11:30 AM, for example based on pharmacokinetic data (such as obtained in clinical trials) and/or based on pharmacodynamic data gathered from a population; in another example, the system estimates that a psychoactive effect induced by the active substance will not influence social abilities after 4 hours from administration; in another example, the system predicts that driving can be safely performed after 3 hours from administration; and the like. Optionally, a prediction or recommendation is provided for a plurality of different activities. For example—you may read work related material in two hours (blood level below a first threshold with a probability of success of 60%) but you can drive only in four hours (blood level below the same or lower threshold, at a probability that is 95% or higher).

In some embodiments, the system (for example via the user interface) issues a notification regarding the predicted effects (403A). The notification may be regarding an effect predicted at one or more times during executing of the delivery regimen. In some embodiments, the system notifies the user regarding a previous effect the delivered substance had on the user (such as during one or more previous deliveries). Optionally, upon being notified about the effects, a user (being the affected user, or another such as a caregiver, a friend or medical staff) may choose to adjust the current regimen, use a different regimen, enter new (or updated) preferences, and/or select, from the listed effects, which are desirable and which are not. In some embodiments, making changes in the regimen is performed in a simple and quick manner, for example via the user interface being programmed to request only minimal input from the user, such as a simple confirmation to change the regimen, entering a password, and the like. In some embodiments the notification may be provided to the user. In some embodiments the notification may be provided to another person, such as a parent and/or a care giver.

In some embodiments, the predicted effects include physiological effects and/or psychoactive effects.

In the example of FIG. 4B, a user is administered the at least one active substance (401B). Optionally, the user is administered the at least one active substance according to the planned delivery regimen. In some embodiments, a system calculates (for example the system processor) an expected blood plasma level of the at least one active substance (403B). Optionally, the expected blood plasma levels are calculated for one or more selected times during or following executing of the delivery regimen (403B). In some embodiments, the expected blood plasma levels for a certain active substance are calculated in accordance with experimental data, such as data obtained during clinical trials. Additionally or alternatively, the expected blood plasma levels for a certain active substance are calculated based on previous assessments of the same user. Optionally, prior effect during use by the user is correlated with clinical trial results showing a correlation between blood concentration and the effect thereby allowing estimation of the user's pharmacokinetic profile without directly measuring it. In some embodiments, data collected from a plurality of users is used as basis for correlating one or more effects (optionally, effects that can be measured non-invasively) with a certain blood plasma level or range. Optionally, based on the correlation and by obtaining input regarding the user (for example voluntary feedback, data acquired by one or more sensors), a user's blood plasma level can be estimated without performing an actual blood test. Optionally, insights derived from AI and/or machine learning, based on user data and/or on data from a plurality of users or user groups, and/or other factors is used to generate the initial regimen and/or adjust it.

In some embodiments, the system (for example via the user interface) notifies the user (or another), regarding the user's expected blood plasma level of the active substance. Optionally, a probability of the blood plasma level being at a certain range is selected (automatically by the system and/or by the user), for example to reach a certainty of 50%, 60%, 75%, 95% 99% or intermediate, higher or lower certainty of the blood plasma level being within a predefined range. In some embodiments an activity is correlated with a blood plasma level and the probability of the blood level is used to estimate the likelihood of the activity being safe, legal, and/or satisfactory. Accordingly, any notification, recommendation, and/or output regarding an activity may include a probability of it being safe, legal, and/or satisfactory as well. The probability assigned to an activity may be the same as that of the correlated blood level. Optionally it may be adjusted according to a degree of correlation between the blood level and the activity being safe, legal, and/or satisfactory.

In some embodiments, adjustment of the regimen is performed in accordance with the blood plasma level timeline. In an example, a system notifies the user that in 30 minutes (or 60 minutes) following a last delivered dose (e.g. 500 μg THC—1,000 μg THC), the user's blood plasma level is expected to be at or below a level suitable for engaging an activity, such as driving.

In some embodiments, the system takes into account a pharmacokinetic and/or pharmacodynamic baseline status of the user, for example when generating the delivery regimen. Optionally, a user enters factual data regarding a previous dose taken (e.g. inhaled a dose of 0.5 mg 50 minutes ago) and/or a degree or state of a psychoactive effect sensed by the user (e.g. on a scale of 1-10). Optionally, the system calculates an expected timeline of the blood plasma level (and/or of other effects) according to the baseline. In some embodiments, the user is presented with the timeline of expected effects and/or with the timeline of predicted blood levels, for example upon initiation of the delivery regimen. In some cases, the user may choose to change one or more planned activities based on the timelines. Optionally an indication is provided via a user interface presenting to the user the likelihood of being below the predefined level.

In some embodiments, the user is presented with an actual blood plasma level. Additionally or alternatively, the user is presented with an associated indication, for example, a schematic graphic indicating to the user that they are within predefined blood plasma level limits. In an example, a smiley or thumbs-up icon is presented when the user is within predefined limits (or over a selected threshold or under a selected threshold). Optionally, the user receives an indication regarding the probability (certainty) of being within selected limits—for example, the smiley turns from red to green when the certainty of being within the selected limits increases, for example from 50% likelihood of being within those limits to an 80% likelihood of being within those limits.

In some embodiments, the system (e.g. the processor) is pre-programmed with one or more thresholds, and the delivery regimen is generated in accordance with the thresholds. In an example, the system is pre-programmed with a maximal blood plasma level, and the regimen is generated accordingly, to prevent the user's blood plasma level from exceeding that threshold.

In some embodiments, pre-defines thresholds are set to reduce or prevent side effects, for example side effects associated with large doses (e.g. dizziness); other thresholds may be set for mitigating counter indications in certain users, for example diabetic users; other thresholds may be set for users with a psychiatric background; and/or due to other reasons or preferences.

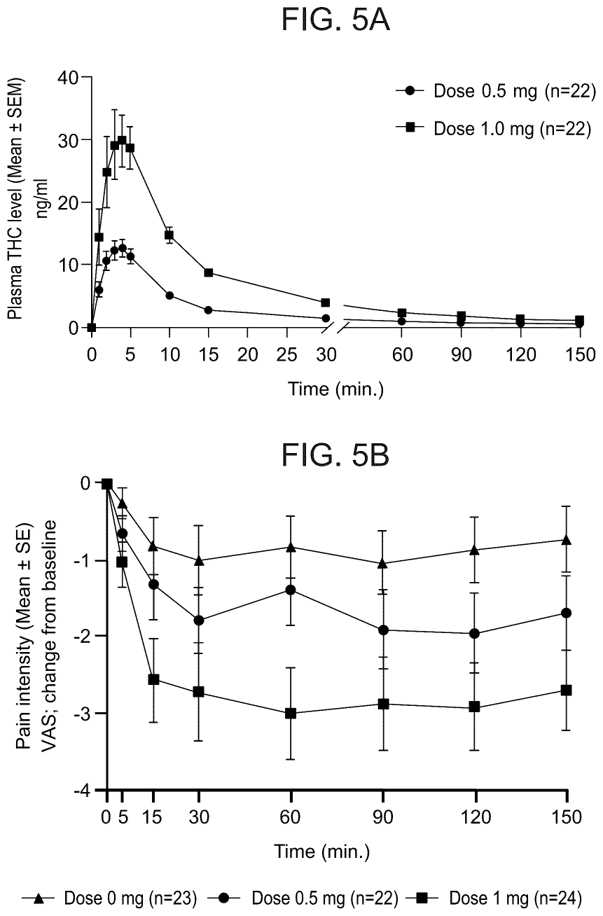

FIGS. 5A-B graphically present results of a clinical trial testing pharmacokinetic effects (5A) and pharmacodynamic effects (5B) of an active substance over time, according to some embodiments.

The results presented herewith are of a clinical trial performed in accordance with some embodiments. An objective of the trial was to evaluate the effects of a single inhalation of vaporized ground cannabis doses comprising 0.5 mg or 1 mg THC, as compared to placebo. THC was administered directly from raw cannabis via a metered dose inhaler device to 27 hospitalized adult patients suffering from chronic pain (exhibiting a baseline of a 0-10 point visual analog scale (VAS) higher than 6). The patients were licensed to use medical cannabis and were guided to refrain from using cannabis at least 12 hours prior to the trial commencement. The patients were guided how to use the inhaler device and signed a written consent.

The trial included three sessions, with an interval of at least 48 hours between sessions. In each of the sessions, a randomized single dose or placebo was delivered to the patients via inhalation. During each session, at a plurality of pre-defined time points, the patients were cognitively tested using “Cambridge Neuropsychological Test Automated Battery”; questioned for assessment of pain (using the VAS); their blood pressure and heartrate were measured; and they were questioned regarding safety parameters, such as feeling “high”, dizziness, mouth dryness. Blood samples were collected at pre-defined time points for performing a pharmacokinetic analysis of THC (being the active substance, in accordance with some embodiments).

FIG. 5A presents comparative plots showing Δ9-THC plasma levels detected following delivery of 0.5 mg Δ9-THC (circular marks) and 1 mg Δ9-THC (square marks). In 22 out of the 27 patients, a pharmacokinetic profile was observed. The remaining patients were not included in the analysis due to non-eligibility, randomization failure and/or technical failure. As shown in the diagram, following single inhalation of 0.5 mg Δ9-THC, the mean maximum plasma Δ9-THC concentration (Cmax) ±SD was 14.3±7.7 ng/ml. The time upon which maximum plasma concentration was reached (Tmax) was 3.7±1.4 minutes, and the mean area under the curve of plasma concentration vs. time from 0 to infinity (AUC_{0→infinity}) was 300±144 ng/min/ml. Following single inhalation of 1 mg Δ9-THC, Cmax ±SD was 33.8±25.7 ng/ml, Tmax was 4.4±2.1 minutes, and the AUC_{0→infinity} was 769±331 ng/min/ml.

FIG. 5B presents effects of the three different interventions on pain intensity. About 1 VAS point decline in pain intensity was noted during the follow-up period following placebo administration. A greater decline in pain intensity was reported following inhalation of 0.5 mg Δ9-THC dose and an even greater decrease following the 1 mg dose. 15 minutes after inhalation and onward, the reduction in VAS was statistically significantly larger in the 1 mg dose as compared to placebo (0 mg) and 0.5 mg (RM-ANOVA, p=0.0015 [95% CI, 0.53; 2.23], p=0.0058 [95% CI, 0.35; 2.08], respectively). A maximum change in VAS pain score in the 0.5 mg dosage was 1.95 points (24%), which was observed at 120 minutes following inhalation, and 2.95 points (39.4%) in the 1 mg dosage, was observed at 60 minutes following inhalation. Diagram 5B demonstrates the changes from baseline in pain intensities at the different time points following the three interventions.

The results of the clinical trial described may support the following indications for automatically planning a delivery regimen, according to some embodiments:
The results show a distinct correlation between the administered dose size and the detected Δ9-THC blood plasma level, and between the administered dose size and the pharmacodynamics effect. Based on this, it is possible to accurately set a delivered amount to one that would achieve a certain plasma level of the active substance and/or certain pharmacodynamics effects or potency thereof.
The results support interpersonal repeatability (for both the pharmacokinetic profile and the pharmacodynamic effect). This suggests that a planned delivery regimen, in accordance with some embodiments, may be suitable for a plurality of users, for example a population having common parameters (e.g. age, medical condition).
A timeline of pharmacodynamic effects and/or a timeline of blood plasma level of the active substance, following delivery of the active substance, may be estimated at a relatively high certainty.

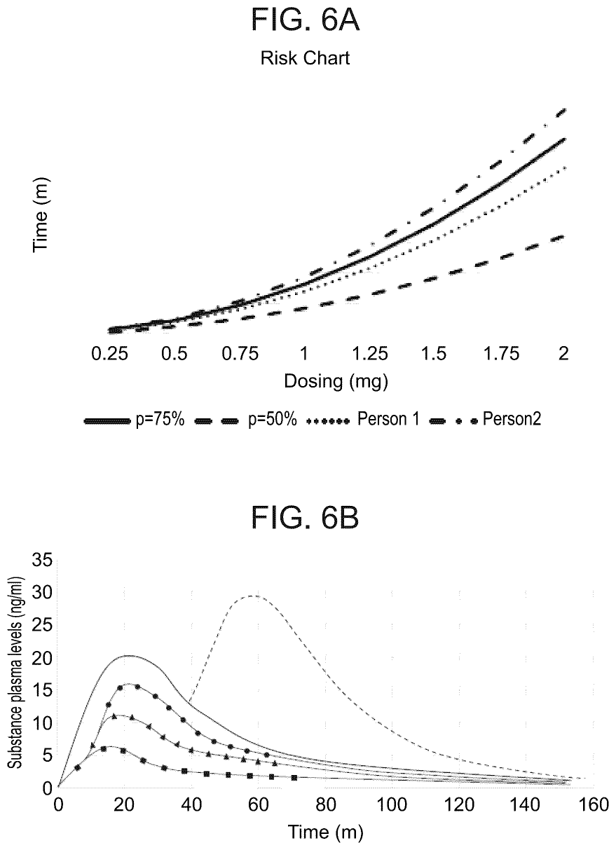

FIGS. 6A-B are: a schematic chart indicating a likelihood of reaching a defined blood plasma level of at least one active substance delivered to a user (6A); and a graph presenting an expected cumulative effect of multiple deliveries of at least one active substance on the blood plasma level (6B), according to some embodiments.

In some embodiments, the delivery regimen is planned by pairing certain times with doses according to probabilities of reaching one or more pharmacokinetic and/or pharmacodynamic effects. Optionally, the probabilities are calculated per the specific user.

In some embodiments, the system receives as input (optionally, inserted by the user) a selected blood plasma level that is to be reached following delivery. Based on the inputted level, the system (for example, the system processor) calculates the probabilities of the time(s) and dose size(s) required to reach the inputted level.

In FIG. 6A a continuous line depicts the times at which there is a 50% chance that the blood plasma level of the at least one active substance would be at the defined level or lower, for different doses of the same substance. A dashed line depicts the times at which there is a 75% chance that the blood plasma level of the at least one active substance would be at the defined level or lower, for different doses of the same substance. For example, a 2 mg dose will have a 50% chance of being below the defined level at about 60 minutes from inhalation, while a little after 120 minutes from inhalation, the probability reaches 75%. Accordingly a desired risk may be selected by the user (or on his/her behalf). In this example Person 1 (dotted line) is satisfied with a surety of a little under 75%, while Person 2 (dashed and dots) required over 75% certainty in her decision making.

In an example, a maximal blood level of an active substance is set to be according to a limitation set by law (for example by regulations) for a given activity, such as at the specified limit or below it. In some embodiments a maximal blood plasma level of THC set, for example, for driving or for operating heavy machinery, may include or be 10 ng THC/ml, 5 ng THC/ml, 3 ng THC/ml, or intermediate, higher or lower levels.

FIG. 6B shows various blood plasma levels based on alternative regimens, as may be estimated by the system according to some embodiments. For example, blood levels of a substance are plotted for a first small dose, as indicated by the line with the square markings. A second similar amount is taken about 10 minutes thereafter and the blood level now estimated according to the first and second doses taken together as indicated by the line with the triangular markings. A third dose is taken at about 17 minutes and the blood levels for the three combined doses is indicated by the line with the circular markings. Optionally, for planning delivery times and/or amounts of the second and/or third doses, the system takes into account the first dose (and/or second dose) delivered, for example, the timing and/or magnitude of one or more effects induced by the previous doses.

In a different regimen, a single dose equaling the total of the three doses of the first regimen is taken and blood levels are plotted as a continuous line. In the example shown, the maximal blood plasma level is reached at a similar timing (Tmax) to that of the first small dose when taken alone, but the value of the maximal blood plasma level is about three times larger than that induced by the first small dose. As further shown in this example, the combined maximal plasma level (Cmax) of the three small doses is smaller than that reached by the single (3X) dose. This may be due to the separation in time of the pharmacokinetic profiles that is meaningful in comparison to the timeline of changes in blood concentration, as schematically shown.

As further shown in this example, a second large dose (having a similar amount of active substance to the first large dose, 3× the single small dose) is taken at 40 minutes and the combined effect of the two large doses (the first taken at time 0 (indicated by the continuous line), and the second taken at 40 minutes) is depicted as the dashed line. Since the second large dose is taken later than the first large dose, the total blood concentration (Cmax, peak of the dashed line) reached is smaller than the level that would have been reached by a single dose comprising twice the amount of the two large doses. As also seen, the total maximal blood level for the three small doses was reached at about 20 minutes, whilst that of the two large doses was reached at about 55 minutes, thus each regimen provided both a different combined Cmax and a different combined Tmax than the other.

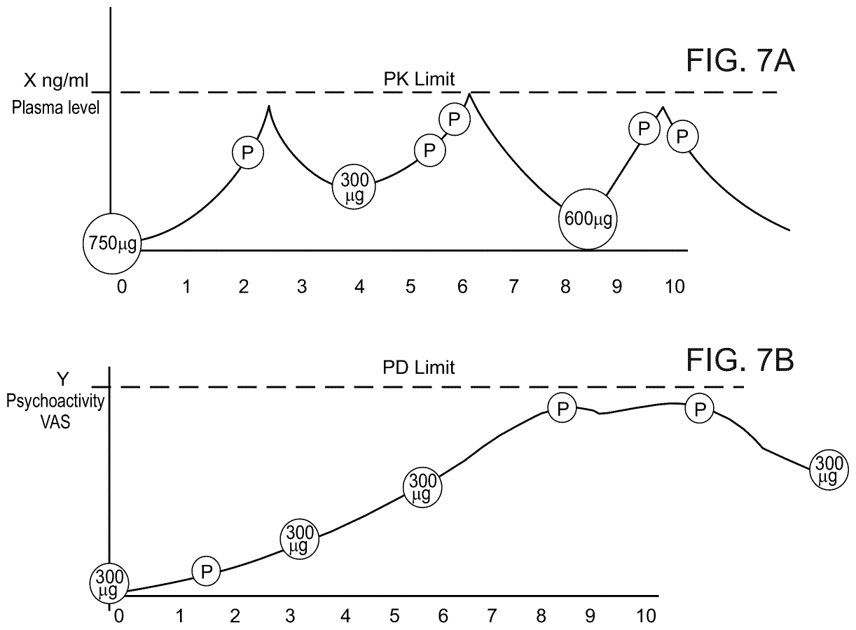

FIGS. 7A-B graphically present examples of delivery regimens planned according to a desired pharmacokinetic limit (7A) and a desired pharmacodynamic limit (7B), according to some embodiments.

FIG. 7A shows an example of a delivery regimen, including different doses provided over time (the dose size is indicated inside the circles). The shown regimen is set with a maximal blood plasma level (generally indicated by “X”) which the regimen is planned not to exceed. The maximal level may be defined according to user preferences, according to regulatory restrictions, according to medical restrictions, and/or other.

FIG. 7B shows an example of a delivery regimen, including different doses provided over time (the dose size is indicated inside the circles). The shown regimen is set with a pharmacodynamic limit, such as a psychoactivity level or effect (generally indicated by “Y”), which the regimen is planned not to exceed. For example, a user defines that they do not want to cross a certain level of feeling “high”.

In some embodiments, as shown in both examples, one or more of the deliveries include placebo (indicated by “P”). Delivering placebo may allow a user to use the delivery device when desired and/or at constant time intervals, without worrying about an unwanted or dangerous effect of additional active dose. A placebo might have the same scent and real time sensation of a dose, with the difference that no active substance (or a very small amount of active substance) is delivered.

In some embodiments, the delivery regimen includes administering of more than one active substance. Optionally, a combination of two or more active substances is provided, at one or more times throughout the delivery regimens.

In an example, active substance(s) are released from different strains of a botanical, such as cannabis.

In some embodiments, a single delivery regimen may combine administering of different cannabinoids, different terpenoids, flavonoid substance and/or placebo. These may be delivered in the same dose, or, alternatively, in separate doses.

In some embodiments the substances are delivered at a regimen based on their combined effect and/or the effect of their combination on the timeline of one or more of their effects. Optionally, at least one substance is delivered to reduce the effect of at least one other substance earlier than it would have reduced otherwise.

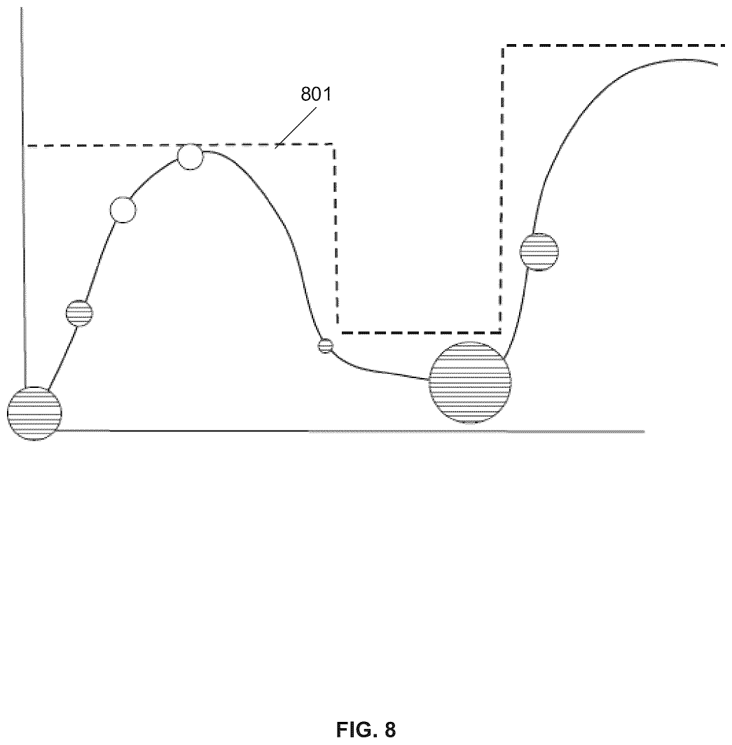

FIG. 8 is an example of a delivery regimen planned to achieve a predefined pattern of limits, according to some embodiments.

In some embodiments, the delivery regimen is planned according to a pattern of limits, schematically indicated by the dashed line 801, including one or both of pharmacokinetic and pharmacodynamic limits. Optionally, minimal and/or maximal limits are set. A different set of limits is applied for different effects and/or active substances.

In some embodiments, the limits are set according to user preferences. Additionally or alternatively, the limits are set based on restrictions, such as regulatory restrictions, medical restrictions, safety restrictions. Additionally or alternatively, the limits are set according to population data. An example of setting limits according to population data may include that a majority of users of a certain population preferred to set a level of an effect, for example a blood plasma level, to a selected value, for example when performing a certain activity or activity type, and then a similar value is set for the specific individual user for when engaged with the same activity or activity type.

As shown in the example of FIG. 8, by setting limits, a shifting “window”, i.e., a range of effect potency, is defined, allowing the user, at each time point along the delivery regimen, to be within the pre-defined limits.

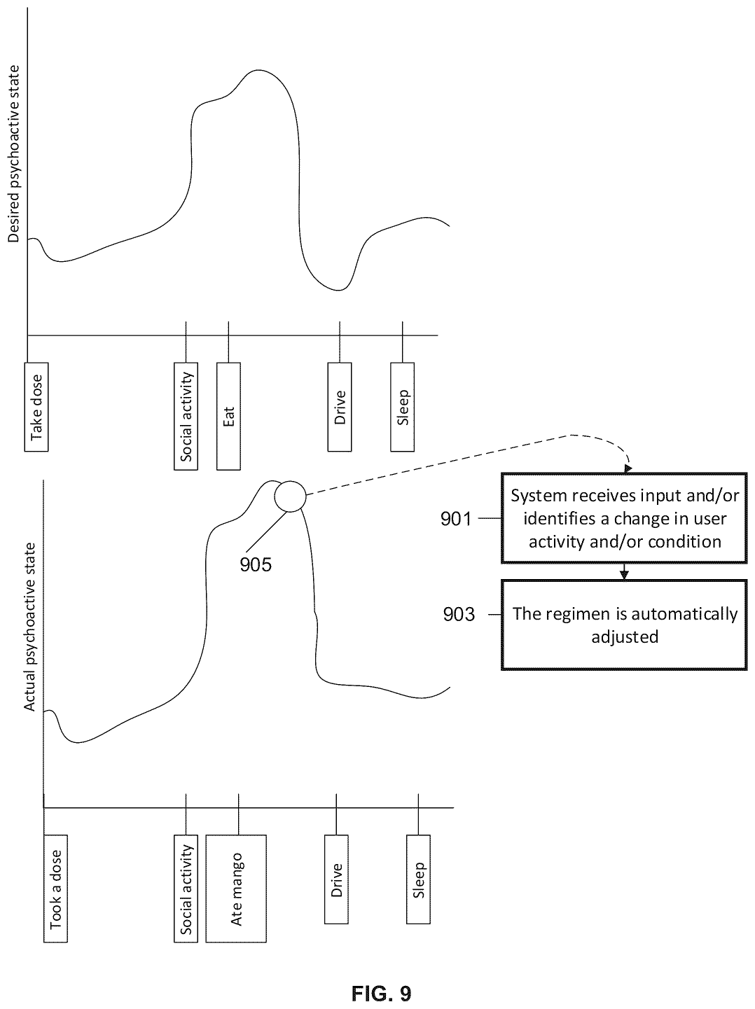

As further shown in the example FIG. 8, different dose sizes and/or types (indicated by the circles) are delivered at different times in order to maintain the user within the selected limits. FIG. 9 is an example of a planned timeline including planned activities and a desired psychoactive state during those activities, and an actual timeline including a change in activity and the manner in which it affects the devilry regimen, according to some embodiments.

The top graph of FIG. 9 schematically illustrates a schedule of planned activities (including, for example, eating, engaging in social activity, driving, sleeping) and a desired psychoactive state during those activities. In some embodiments, the scheduled activities and the paired desired psychoactive state are inserted as input into the delivery regimen planning system. Optionally, the user takes an initial dose of an active substance, such as THC, before the planned activities.

In some embodiments, during execution of the delivery regimen, one or more changes occur, for example, a change in the scheduled activities or a timing thereof; a preferred change in the psychoactive state; and/or other changes which may affect the initially planned delivery regimen. In the bottom graph of FIG. 9, a change in activity which optionally affects the psychoactive state occurs (see 905). In the shown example, the change in activity involves that the user, when eating, eats mangoes (possibly without planning the eating of mangoes ahead of time) at a substantial amount, for example an amount equivalent to two mangoes. Since mango at a certain amount is known to enhance the effects of THC (for example enhancing the effect twofold), presumably as it includes a terpene known as “myrcene”, consumption of mango by the user may require an adjustment of the planned regimen. Adjustment of the regimen may include, for example: a reduction of the next dose (e.g. reducing THC of the next dose by 50%), delaying the delivery of the next dose (e.g. by an hour), and/or setting delivery of one or more active substances (such as CBD) which may counteract the psychoactive side effects (such as anxiety). In the example shown, after consuming mango, a user's psychoactive state is expected to rise to a higher level than may be desired.

In another example, a substance that affects the psychoactive state of the user is administered or consumed. For example, the user drinks alcohol. In such situation, the delivery regimen may be adjusted, while optionally taking into account the amount and/or concentration of the substance (e.g. alcohol), and/or the timing of consumption. Optionally, the adjustment is performed based on a known and/or estimated effect of the substance consumed. In some embodiments, the adjustment is performed based on previous effects of the same substance on the specific user.

Then, in some embodiments, the system receives input from the user (for example, the user or another on his behalf inserts that the user consumed mango at a substantial amount) and/or automatically identifies the change in the user's condition (for example via one or more sensors, by having the user perform a mental and/or physical task such as playing a game on the user interface, and/or other detection methods (for example, by communication with a bar's billing system) (901).

Based on the received and/or detected change, the system (e.g. the system processor) re-calculates the delivery regimen and optionally adjusts it in view of the change (903). Optionally, the adjustment is calculated taking into account a current condition, for example that the user had reached a higher psychoactive level than desired. Optionally, the adjustment is calculated taking one or more upcoming activities into account. In this example, where an upcoming activity involves driving, the system may reduce, prevent (e.g. by automatically locking), or deliver placebo instead of the next originally planned dose, so as to bring the user to a psychoactive level which is low enough to be suitable (and legal) for driving. The regimen may be further adjusted to bring the user to a desired psychoactive state before or during sleep.

In another example, a user consumes another active substance, for example takes a medication, which may induce a psychoactive effect when used on its own and/or when combined with another substance, such as THC. In a specific example, the medication is methylphenidate hydrochloride, which is expected to affect a user's psychoactive state, when used independently and/or when taken with, before and/or after THC. In such situation, the system estimates a combined effect of the multiple active substances consumed, and optionally that effect is taken into account when generating the delivery regimen.

Other examples that in which a regimen may be set to address a combined psychoactive effect of different substances may include consumption of alcohol, taking short term psychoactive medication, smoking a cannabis cigarette (optionally in addition to inhalation of THC from the inhaler device) and/or other, in which case the regimen may be adjusted to skip one or more doses of THC, add or increase CBD to the dose, and/or other adjustments which may counteract undesired side effects.

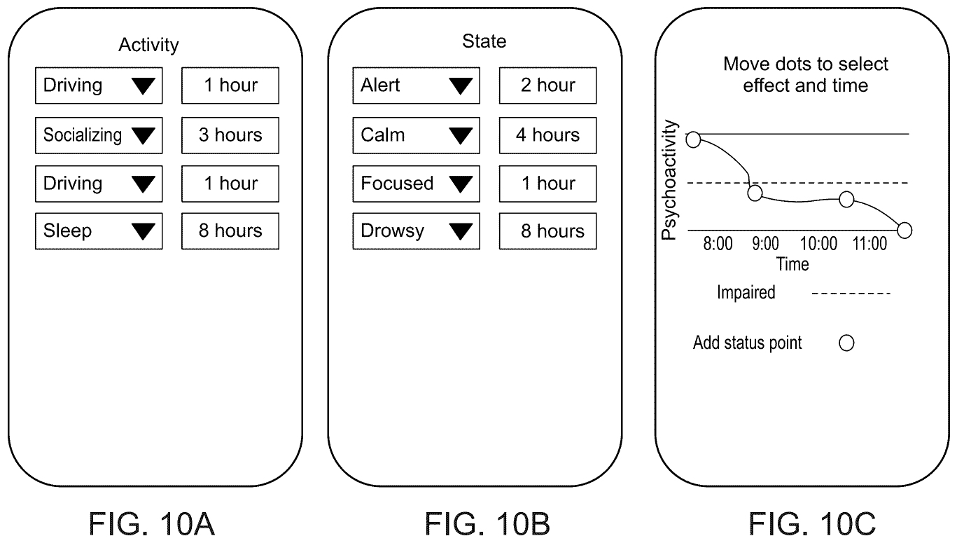

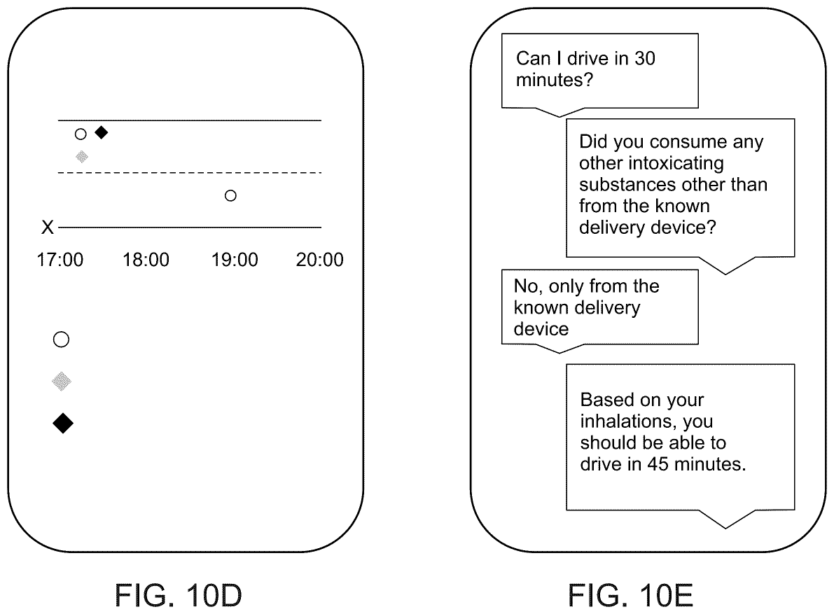

FIGS. 10A-E are examples of user interface screens of an active substance delivery planning application, according to some embodiments.

In some embodiments, a dedicated software application is used, installed for example on the user's personal digital device (e.g. cell phone, tablet computer).

Following are examples of features that may be included in such application. In the example screen of FIG. 10A, the user selects one or more planned activities, and a duration and/or starting time for each of the activities. The selection may indicate the order of the activities. In the example screen of FIG. 10B, the user selects a preferred user experience, for example a mental state and/or a physical state, associated with the time and/or with the previously indicated activities. It is noted that time may be indicated in different manners, such as: the time until start, the specific hour in the day, a duration (e.g. of an activity and/or a preferred mental state and/or a preferred physical state).

In the example screen of FIG. 10C, a plot showing an effect of the active substance, such as a psychoactivity level, is presented. The plot may be generated according to input previously inserted, for example associate between the activities and the mental/physical states (e.g. FIG. 10A and FIG. 10B) on a timeline. Optionally, the user may use the interface to adjust one or more points, for example by setting a different time and/or psychoactivity level. Optionally, in this example the plot is provided on a touchscreen and the user may move, add and/or remove the points which represent a desired effect. In some embodiments, the user receives a plot and marks the different points directly on the timeline, (optionally without inputting data on the previous screens). In some embodiments, the user adjusts (or draws in) one or more bars (see the dashed line) defining for example a preferred degree of psychoactivity, optionally indicating a maximal and/or minimal level desired. Optionally, one or more bars are provided or suggested by the system automatically to denote a blood level below which driving is permitted or a blood level above or below which a given effect is expected. Such one or more bars may be adjusted by the system in time, taking into account data received, for example regarding the user's sensitivity or resistance and/or past use. In some embodiments, a pharmacokinetic plot is presented, for example showing blood plasma concentration levels of the active substance. Such plot may be presented on the same timeline as the psychoactivity plot, or separately.

In the example screen of FIG. 10D, a user inputs a request, including, for example, a preferred user experience (optionally varying over time), a preferred amount of active substance (such as to be provided in a single use or a total to be provided over multiple uses), a preferred timing of delivery, planned activities, and/or other requests. The system attempts to generate a delivery regimen which best addresses the request. In some cases, the request cannot be fulfilled—for example, if a user request an amount that is not safe; if a user requests that deliveries will be timed too close to each other, considering the amount being delivered; if a user requests a user experience which cannot be carried out; if the user's planned activities contradict the amounts being delivered (for example—an amount being delivered is too high for the user to be able to drive), if the pharmacokinetic timeline does not support the request; etc. In such cases, the system can generate one or more alternative regimens (indicated as “option A” and “option B”). The user may select one of the proposed options, ask the system to generate another option, and/or modify the request.

In the example screen of FIG. 10E, the user communicates with the interface by natural text (or optionally, voice). Optionally, the user asks specific questions, such as “can I drive in 30 minutes” ? and the interface provides the required information, optionally after further interrogation of the user (e.g. the system checks if another active substance was consumed by the user).

Optionally a screen is shown by the system depicting the user's status in real time, without being interrogated by the user (such as a default home screen when turned on during a regimen). In some embodiments, the screen shows the user's expected active substance blood level, their expected effect or a warning, such as “Please do not drive”.

According to some embodiments, the source material from which the at least one active substance is released comprises botanical material, including, for example: Cannabis sativa, Cannabis indica, Cannabis ruderalis, Acacia spp, Amanita muscaria, Yage, Atropa belladonna, Areca catechu, Brugmansia spp., Brunfelsia latifolia, Desmanthus illinoensis, Banisteriopsis caapi, Trichocereus spp., Theobroma cacao, Capsicum spp., Cestrum spp., Erythroxylum coca, Solenostemon scutellarioides, Arundo donax, Coffea arabica, Datura spp., Desfontainia spp., Diplopterys cabrerana, Ephedra sinica, Claviceps purpurea, Paullinia cupana, Argyreia nervosa, Hyoscyamus niger, Tabernanthe iboga, Lagochilus inebriens, Justicia pectoralis, Sceletium tortuosum, Piper methysticum, Catha edulis, Mitragyna speciosa, Leonotis leonurus, Nymphaea spp., Nelumbo spp., Sophora secundiflora, Mucuna pruriens, Mandragora officinarum, Mimosa tenuiflora, Ipomoea violacea, Psilocybe spp., Panaeolus spp., Myristica fragrans, Turbina corymbosa, Passiflora incarnata, Lophophora williamsii, Phalaris spp., Duboisia hopwoodii, Papaver somniferum, Psychotria viridis, spp., Salvia divinorum, Combretum quadrangulare, Trichocereus pachanoi, Heimia salicifolia, Stipa robusta, Solandra spp., Hypericum perforatum, Peganum harmala, Tabernaemontanaspp, Camellia sinensis, Nicotiana tabacum, rusticum, Virola theidora, Voacanga africana, Lactuca virosa, Artemisia absinthium, Ilex paraguariensis, Anadenanthera spp., Corynanthe yohimbe, Calea zacatechichi, Coffea spp. (Rubiaceae), a Sapindaceae, Camellia spp., Malvaceae spp., Aquifoliaceae spp., Hoodia, spp. Chamomilla recutita, Passiflora incarnate, Camellia sinensis, Mentha piperita, Mentha spicata, Rubus idaeus, Eucalyptus globulus, Lavandula officinalis, Thymus vulgaris, Melissa officinalis, Aloe Vera, Angelica, Anise, Ayahuasca (Banisteriopsis caapi), Barberry, Black Horehound, Blue Lotus, Burdock, Camomille/Chamomile, Caraway, Cat's Claw, Clove, Comfrey, Corn Silk, Couch Grass, Damiana, Damiana, Dandelion, Ephedra, Eucalyptus, Evening Primrose, Fennel, Feverfew, Fringe Tree, Garlic, Ginger, Ginkgo, Ginseng, Goldenrod, Goldenseal, Gotu Kola, Green Tea, Guarana, Hawthorn, Hops, Horsetail, Hyssop, Kola Nut, Kratom, Lavender, Lemon Balm, Licorice, Lion's Tail (Wild Dagga), Maca Root, Marshmallow, Meadowsweet, Milk Thistle, Motherwort, Passion Flower, Passionflower, Peppermint, Prickly Poppy, Purslane, Raspberry Leaf, Red Poppy, Sage, Saw Palmetto, Sida Cordifolia, Sinicuichi (Mayan Sun Opener), Spearmint, Sweet Flag, Syrian Rue (Peganum harmala), Thyme, Turmeric, Valerian, Wild Yam, Wormwood, Yarrow, Yerba Mate, Yohimbe, Galanthus woronowii, Rauvolfia reserpine, Ginkgo biloba, Boswellia papyrifera, Centella asiatica, Rosmarinus officinalis, Eschscholtzia californica, Abutilon indicum, Bacopa Monnieri, Rhodiola Rosea, Huperzia Serrata, Erythrina mulungu, Hippeastrum vittatum, Afzelia Africana, Crocus sativus, Punica granatum, Physostigma venosum, Geissospermum vellosii, Salsola oppositefolia, Mexican peyote cactus, Gastrodia elata, Narcissus spp., Magnoliaceae spp., and any part and any combination thereof.

According to some embodiments, the botanical material includes plant material, including for example Cannabis sativa, Cannabis indica, and Cannabis ruderalis.

According to some embodiments, the active substance includes a cannabinoid or an acidic form of a cannabinoid. Examples include Δ9-tetrahydrocannabinol (THC), cannabidiol (CBD), cannabigerols (CBG), cannabichromenes (CBC), cannabinol (CBN), cannabinodiol (CBDL), cannabicyclol (CBL), cannabielsoin (CBE), cannabidivarin (CBDV), tetrahydrocannabivarin (THCV) and cannabitriol (CBT) and acidic forms of the aforementioned.

According to some embodiments, the active substance includes Δ9-tetrahydrocannabinol (THC), tetrahydrocannabinolic acid (THCA), cannabidiol (CBD) and/or cannabidiolic acid (CBDA).

According to some embodiments, the active substance comprises legally approved non-medical drugs, such as caffeine, cathinone, cathin, nicotine, Myristicin (Methoxysafrole), trans-neoclerodane diterpenoids, such as salvinorin A, sesquiterpene lactones, such as germacranolides, dextromethorphan, 4-ACO-DMT (also known as O-Acetylpsilocin or psilacetin), kavalactones (or kavapyrones), such as kavain, dihydrokavain, methysticin, dihydromethysticin, yangonin, and desmethoxyyangonin; active substances present in Turnera diffusa, active substances present in Argyreia nervosa, such as ergot alkaloids; active substances present in Echinopsis pachanoi and/or in Lophophora williamsii, such as mescaline, 3,4-dimethoxyphenethylamine, 3-Methoxytyramine, 4-hydroxy-3-methoxyphenethylamine, 4-hydroxy-3,5-dimethoxyphenethylamine, anhalonidine, anhalinine, hordenine, and tyramine; aporphine; active substances present in Amanita muscaria, such as muscarine, ibotenic acid, muscimol and Muscazone; hallucinogenic tryptamines present in Incilius alvariusvenom venom, such as 5-MeO-DMT and Bufotenin

According to some embodiments, the active agent is or includes a terpenoid, alkaloid or cannabinoid. For example, in some embodiments, the active agent is a diterpenoid such as, but not limited to salvinorin A from Salvia. In other embodiments, the active agent is an alkaloid such as, but not limited to, benzoylmethylecgonine from the coca plant, or the active agent is a tryptamine such as psylocibin from mushrooms. In alternative embodiments the active substance is dimethyltryptamine (DMT) from a variety of botanicals. In further embodiments, the active substance is nicotine from tobacco. In further embodiments, the active substance is a terpenoid, e.g., limonene, α-pinene, β-myrcene, linalool, β-caryophyllene, caryophyllene, nerolidol or phytol, present in various botanical (e.g. plant) forms.

Substances (from an organic source or from a non-organic source) which have medicinal properties and are suitable for use with delivery systems and/or devices and/or methods for example as described herein are also to be contemplated by this application.

As referred to herein, the term pharmacokinetic effect (or profile) may refer to a measurable (optionally quantifiable) physiological effect in a user, which pertains to the presence of an active substance in a user. Pharmacokinetic effects are direct or indirect expressions of a group of physiological processes that include absorption, distribution, metabolism, and excretion of an active substance in a user.

Pharmacokinetic effects typically include, without limitation:
The concentration of an active substance, as determined, measured or assessed in a specific physiologic system (e.g., in the plasma), after its administration (delivery) of a dose or a regimen to a user;
The peak concentration of an active substance, as determined, measured or assessed in a specific physiologic system (typically in the plasma), after its administration to the user (Cmax);
The time passed between administration and arriving at the peak concentration (Tmax);
An area under the curve (AUCO→∞; zero to infinity), which is the integral of the concentration curve as a function of time, typically after a single dose or in steady state;
The lowest concentration of the active substance in the organism before the next dose is administered;
The time passed until the lowest concentration is detected, or until the next dose is administered;
The last observed quantifiable concentration;
And/or other parameters.

As referred to herein, the term “pharmacodynamic effect” (or profile) may refer to a group of effects pertaining to a user and an active substance, which are manifested in the user upon administering the substance. Typically, pharmacodynamic effects depend on the user's pharmacokinetic effects.

Pharmacodynamic effects may include a therapeutic effect, an adverse effect, a psychoactive state, physiological effects or sensations, The pharmacodynamic effect, in some embodiments, is a personally perceived effect.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments may be presented in a range format. It should be understood that the description in range format is merely for convenience and brevity and should not be construed as an inflexible limitation on the scope of an invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition

It is appreciated that certain features of an invention, which are, for clarity, described in the context of separate embodiments, may also be provided in combination in a single embodiment. Conversely, various features of an invention, which are, for brevity, described in the context of a single embodiment, may also be provided separately or in any suitable subcombination or as suitable in any other described embodiment of an invention. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method for generating a delivery regimen of at least one active substance to a user, comprising:
receiving input including one or both of timed activities of the user, timed preferred user experiences;
and
automatically generating using a processor a delivery regimen including at least one of delivery times and amounts of the at least one active substance, based on:
(a) estimating at least one of a timing and magnitude of at least one effect expected to be induced by the at least one active substance on the user; and
(b) estimating an impact of the at least one effect on the timed activities and/or the timed preferred user experiences.

2. A method for generating a delivery regimen of at least one active substance to a user via inhalation, comprising:
receiving input including one or both of timed activities to be engaged by the user, timed preferred user experiences; and
automatically generating a delivery regimen including at least one of delivery times and amounts of the at least one active substance, based on an expected timeline of a blood plasma level of the at least one active substance and based on an estimated impact of the blood plasma level on one or both of the timed activities and the timed preferred user experiences.

3. A method for generating time-based recommendations to a user, comprising:
receiving input including a timing and amount of one or more doses of at least one active substance previously taken by the user and/or one or more planned doses; and
automatically generating a time-based recommendation based at least partially on estimating at least one of a timing and magnitude of at least one effect expected to be induced by the at least one active substance on the user, the time-based recommendation including an indication of the user's capability for engaging in one or more activities, wherein the capability is correlated with the at least one effect; and
outputting the recommendation.
